(19) United States
(12) Patent Application Publication
(10) Pub. No.: US 2024/0317934 A1
(43) Pub. Date: Sep. 26, 2024

(54) POLY(ETHYLENE GLYCOL) HAVING C1 TO C3-ALKYLOXYMETHYL SIDE CHAINS, BIOCONJUGATES THEREOF, PROCESS FOR ITS PREPARATION AND ITS USE

(71) Applicant: JOHANNES GUTENBERG-UNIVERSITÄT MAINZ, Mainz (DE)

(72) Inventors: Holger FREY, Mainz (DE); Rebecca MATTHES, Mainz (DE); Philip DREIER, Mainz (DE)

(73) Assignee: JOHANNES GUTENBERG-UNIVERSITÄT MAINZ, Mainz (DE)

(21) Appl. No.: 18/290,047

(22) PCT Filed: May 12, 2022
(86) PCT No.: PCT/EP2022/062896

§ 371 Date: Nov. 9, 2023

(30) Foreign Application Priority Data
May 14, 2021 (EP) 21173944.6

(51) Int. Cl. C08G 65/333; A61K 39/00; A61K 39/215; A61K 47/60; C08G 65/22

(52) U.S. Cl. CPC C08G 65/33396; A61K 39/215; A61K 47/60; C08G 65/22; A61K 2039/6018; A61K 2039/6093

(57) ABSTRACT

Polyether polymers represented by the following formula [I]:

—(CH₂CHR—O—)ₘ— (I)

wherein 10 to 90% of the residues R are hydrogen and 10% to 90% of the residues R are independently from each other selected from the group made of, methoxymethyl, ethoxymethyl, n-propoxymethyl and iso-propoxymethyl; 1 to 100% of the residues R are methoxymethyl; up to 50% of the residues R may be selected from the group made of ethoxymethyl, n-propoxymethyl and iso-propoxymethyl; and m is in the range of from 10 to 1000 and the dispersity is 1.15 or less. A process for their preparation, conjugates thereof and the use thereof.

The present invention concerns polyether polymers represented by the following formula [I]:

—(CH₂CHR—O—)ₘ— [I]

wherein 10 to 90% of the residues R are hydrogen and 10% to 90% of the residues R are independently from each other selected from the group consisting of, methoxymethyl, ethoxymethyl, n-propoxymethyl and iso-propoxymethyl; 1 to 100% of the residues R are methoxymethyl; up to 50% of the residues R may be selected from the group consisting of ethoxymethyl, n-propoxymethyl and iso-propoxymethyl; and m is in the range of from 10 to 1000 and the dispersity is 1.15 or less.

The invention further concerns a process for their preparation, conjugates thereof and the use thereof.

### BACKGROUND OF THE INVENTION

The aliphatic polyether poly(ethylene glycol) (PEG) is the most widely employed polymer in pharmaceutics, medicine and drug delivery. PEGylation, i.e. the attachment of PEG to peptide drugs, was introduced in the 1970ies and has since then led to numerous highly efficient drugs. PEGylation is a form of conjugation, i.e. the attachment of a polymer to a molecule by a covalent bond. The main effect of the attachment of PEG chains is the so-called “stealth effect”, which prevents close approach of and recognition by the immune system and subsequent binding and removal by the reticuloendothelial system (RES) in the liver. More simply, PEG attachment also prevents opsonization by proteins, when attached to the surfaces of nanocarriers for drug delivery. The inhibition of protein adsorption by PEG is further emphasized by fulfillment of the rules for protein-repellent surfaces derived by Whitesides (Yarovsky et al., “Quantitative design rules for protein-resistant surface coatings using machine learning”, Scientific Rep. 2019, 9, 265). PEGylation is also used for “stealth liposomes” with long circulation times, low molecular weight drugs as well as for lipid nanoparticles. Such structures are employed for the transport and delivery of mRNA. Here the respective PEG-based lipids play a key role for the vaccination against the COVID-19 pandemic, enabling transport of mRNA to cells, avoiding undesired degradation processes. The recently approved mRNA vaccines from Moderna and Pfizer-BioNTech require PEGylated lipids to ensure the stability of the mRNA in the lipid-based nanoparticle. PEGylation also prevents coalescence of liposomes and similar structures in aqueous systems. Commonly, molecular weights in the range of 2 to 40 kg/mol are preferred for the PEGylation of drugs or nanocarriers. To sum up, PEG has become a standard for water-soluble medical polymers for a vast variety of medical and pharmaceutical uses, particularly in the therapy of cancer, chronic diseases and recently also for RNA delivery vehicles. The success of PEG may have had several reasons. Among them are commercial availability in sufficient quality and in a wide range of molar masses from the anionic ring-opening polymerization of ethylene oxide as well as its extremely low toxicity and high biocompatibility. Further, PEG can be prepared in narrow molecular weight distributions.

However, an increasing number of studies summarize concerns related to the presence of so-called anti-PEG antibodies (APAs) that are present in a large and constantly growing part of the population. Such antibodies can lead to accelerated clearance of PEGylated drugs from the blood stream, i.e. loss of the stealth effect that is crucial for therapeutic success, allergic reactions and in extreme and rare cases even potentially fatal anaphylactic shock. This undesired effect is observed both for PEGylated peptide drugs, PEGylated liposomes and other PEGylated nanocarriers as well as PEGylated small molecule drugs. Anti-PEG antibodies are also problematic with respect to PEGylated lipids for RNA-transporting lipid nanoparticles, e.g. in the context of COVID-19 vaccines. PEG-antibodies are often mentioned in the context of potential PEG-alternatives as a motivation to look beyond PEG for alternatives. As an estimate, approximately 10% of the population experience allergic reactions when treated with PEGylated formulations. Only recently, the nature of the binding between PEG and anti-PEG antibodies has been studied by Lai et al (Lai et al, “Structure of an anti-PEG antibody reveals an open ring that captures highly flexible PEG polymers.” Commun Chem 2020, 3, 124). They concluded that binding of PEG is due to an open ring structure of PEG captured by the APAs. By counting the number of monomer repeats of the PEG polymer interacting with the interior and exterior paratope of the Fab, they found the size of the PEG antigen epitope to be ˜700 g/mol, equivalent to 16 monomer subunits. Thus, at least 16 ethylene glycol units are required for binding by the APA. Other works suggest that also shorter regular chain segments of PEG as well as the methoxy end group of mPEG (α-methoxy poly(ethylene glycol)) structure play a key role for the recognition. U.S. Pat. No. 8,129,330 B2 therefore discloses methods for the preparation of PEG conjugates that are based on end group modification of the PEG chains. Variation of the end group of PEG is disclosed, aiming at minimizing interaction of for example methoxy end groups with anti-PEG antibodies. The authors disclose conjugates, wherein a hydroxyl group is present on all of the distal polyalkylene glycol termini in said pure conjugate, and wherein said conjugate exhibits reduced antigenicity compared to mPEG-protein conjugates. It has been reported that antibodies elicited by PEG-OH have similar affinity to both mPEG and PEG-OH, while antibodies induced by mPEG recognize mPEG more effectively than PEG-OH (Sherman et al, “Role of the methoxy group in immune responses to mPEG-protein conjugates.” Bioconjug Chem 2012, 23(3), 485-499; Sherman et al, “Selectivity of binding of PEGs and PEG-like oligomers to anti-PEG antibodies induced by methoxyPEG-proteins.” Mol Immunol 2014, 57(2), 236-246.). These results have been obtained by using competitive ELISA and imply that the anti-PEG antibodies elicited by PEG-OH-proteins are directed against the backbone of the PEG (backbone-specific), while antibodies induced by mPEG-protein conjugates are methoxy group specific. It can be concluded that regular segments of at least 4-5 to 16 regularly arranged ethylene glycol units are required to achieve recognition and immunogenic reaction by anti-PEG antibodies. Recent clinical works demonstrated that the presence of antibodies against PEG permits to predict Pegasparagase allergic reactions and failure of rechallenge, emphasizing the clinical importance of anti-PEG antibodies for the success of the treatment of leukemia in this case (Liu et al, “Antibodies Predict Pegasparagase Allergic Reactions and Failure of Rechallenge”, J. Clin. Oncol. 2019, 37, 2051).

Three strategies for the modification of the PEG structure have been developed to counteract undesired APA interaction of PEGylated therapeutics. Replacing the methoxy end group of mPEG by a hydroxyl group led to a strong decrease in the affinity for anti-PEG antibodies, which was demonstrated by competitive ELISA tests. However, it is obvious that for large scale commercial applications the presence of a hydroxyl group at the terminus would require protected functional initiators, which complicates existing synthetic protocols. A second recent strategy is disclosed in US 2019/0015520 A1 and relies on PEG-bottlebrush structures, i.e. small PEG structures appended to a poly(hydroxyethyl methacrylate) backbone. The inventors state that this type of PEG-architecture minimizes anti-PEG antigenicity, while preserving the desired stealth properties for surface coating. However, the resulting PEG-grafted copolymers exhibit rather broad, multimodal molecular weight distributions, which represent a major obstacle for approval for medical application and PEGylation analogous bioconjugation with peptides or lipids. Narrow distributions (Mw/Mn<1.15) were exclusively feasible by performing a cost-and time-consuming preparative size-exclusion chromatography (SEC).

Another strategy is to exchange PEG with the linear polyether polyglycerol to increase the blood circulation time of liposomes and to avoid accelerated blood clearance (see Abu Lila et al, “Use of polyglycerol (PG), instead of polyethylene glycol (PEG), prevents induction of the accelerated blood clearance phenomenon against long-circulating liposomes upon repeated administration”, International Journal of Pharmaceutics, 456 (2013) 235-242). However, the utilization of polyglycerol also results in the introduction of multiple hydroxyl functionalities to the liposome, which is in contrast to the aforementioned rules of Whitesides resulting in complex unspecific recognitions of peptides in the blood stream. Therefore, a targeted use of the conjugated drug or liposomes is not possible with polyglycerol.

Since PEGylated therapeutics are often used for chronic or in many cases otherwise fatal diseases, like in cancer therapy, in many cases repeated doses have to be administered over prolonged periods of time. PEG is a non-biodegradable polymer and has to be eliminated from the body through the kidneys. In studies on the renal filtration of PEGylated drugs, an undesirable accumulation of PEG in the kidneys and the associated pathological changes in the renal system could be demonstrated (see e.g. Bendele, A.; Seely, J.; Richey, C.; Sennello, G.; Shopp, G. Short Communication: Renal Tubular Vacuolation in Animals Treated with Polyethylene-Glycol-Conjugated Proteins. Toxicol. Sei. 1998, 42, 152-157). The tendency of polyethylene to crystallize can therefore lead to deposits in the kidney. The elimination through the kidneys also limits the molecular weight of PEG for medical therapeutics to the renal cutoff size, which for globular proteins is approximately 50 to 60 kDa.

The present inventors have reported in 2014 on the copolymerization of ethylene oxide with glycidyl methyl ether (GME) by use of the monomer activation method and tetraoctylammonium bromide and tris-iso-butylaluminum as initiators (see Frey et al, A Challenging Comonomer Pair: “Copolymerization of Ethylene Oxide and Glycidyl Methyl Ether to Thermoresponsive Polyethers”, Macromolecules 2014, 47, 5492-5500). Rather broad molecular weight distributions were obtained (dispersity in the range of 1.21 to 1.5), which are clearly not suitable for medical applications. As discussed at the time, with the conventional anionic ring-opening method only molecular weights up to 3000 g/mol and high dispersities have been reported for the polymerization of GME. In previous studies regarding the homopolymerization of GME to poly(glycidyl methyl ether) (PGME) it was generally stated that polymerization causes difficulties, and no molecular weight control can be achieved by anionic ring-opening polymerization as employed for medical PEG (Frey et al., Biomacromolecules 2014, 15, 1935-1954). In all available reports regarding GME polymerization to date, either phosphazene bases were used as a catalyst or aluminum compounds had to be added in the way of a “monomer activated polymerization”. The same applies to the reaction as published by Labbé et al. (Macromol. Symp. 2007, 249-250, 392-397) using monomer-activated ring-opening polymerization for synthesis of polyglycidylether. This technique suffers from a common side reaction described in FIG. 11. The undesired transfer of hydride leads to further initiating species, therefore limiting the required high end-group fidelity. Additionally, the occurrence of initiation by iso-propyl via monomer-activated ring-opening polymerization is also reported in the literature e.g. in Billouard, C.; Carlotti, S.; Desbois, P.; Deffieux, A. “Controlled” High-Speed Anionic Polymerization of Propylene Oxide Initiated by Alkali Metal Alkoxide/Trialkylaluminum Systems. Macromolecules 2004, 37 (11), 4038-4043. DOI: 10.1021/ma035768t and Herzberger, J.; Niederer, K.; Pohlit, H.; Seiwert, J.; Worm, M.; Wurm, F. R.; Frey, H. Polymerization of Ethylene Oxide, Propylene Oxide, and Other Alkylene Oxides: Synthesis, Novel Polymer Architectures, and Bioconjugation. Chemical reviews 2016, 116 (4), 2170-2243. DOI: 10.1021/acs.chemrev.5b00441. None of these polymers or copolymers are suitable for medical applications for several reasons: (i) traces of phosphazene bases lead to toxicity and cannot be removed; (ii) dispersities exceed 1.1, which is prohibitive for most medical applications and (iii) end-group fidelity of the polymers derived from the methods is insufficient for attachment to peptides, drugs or lipids via bioconjugation. Therefore, neither PGME homopolymer nor copolymers of GME have been considered for PEGylation type applications to date.

The formation and retention of well-defined polymer end-groups (end-group fidelity) is a critical tool for chain extension and all post polymerization reactions. End-group fidelity is also a decisive factor for the use of polymers in bioconjugates for pharmaceutical applications, in which the biomolecules are connected by means of the end-groups. Polymers with low end-group fidelity comprise a large amount of macromolecules that are not bound to the biomolecule and cannot be removed from the mixture without undue expenditure. The formed conjugates do not have the required purity. The loss of end-groups is therefore not desirable but cannot be completely avoided. End-group fidelity varies greatly depending on the reaction conditions in the preparation of the polymers.

### Purpose of the Invention

It is the objective of the present invention to provide alternative polymers that are able to replace PEG in its applications and that can alleviate at least some of the aforementioned disadvantages of PEG. The alternative polymer should especially be suited to replace PEG in pharmaceutical applications. It is further an objective to provide polymers with low dispersity. A further objective is to provide polymers with high end-group fidelity. It is also desired to provide a process that allows preparation of such polymers in a simple and commercially feasible way. The alternative polymers should have a low affinity towards anti-PEG antibodies (APAs). A further goal is to provide polymers with a low immunogenicity.

### DETAILED DESCRIPTION

The present invention concerns poly(ethylene glycol) having C1 to C3-alkyloxymethyl side chains. The present invention concerns polyether polymers represented by the following formula [I]:

—(CH₂CHR—O—)ₘ— [I]

wherein 10 to 90% of the residues R are hydrogen and 10 to 90% of the residues R are independently from each other selected from the group constisting of methoxymethyl ethoxymethyl, n-propoxymethyl and iso-propoxymethyl; 1 to 100% of the residue R are methoxymethyl; up to 50% of the residues R may be selected from the group consisting of ethoxymethyl, n-propoxymethyl and iso-propoxymethyl; with the proviso that at least one residue R is hydrogen, if at least one residue R is selected from the group consisting of ethoxymethyl, n-propoxymethyl and iso-propoxymethyl; and m is in the range of from 10 to 1000, characterized in that the dispersity is 1.15 or less measured by size exclusion chromatography in DMF by calibration with PEG standards. Herein the percentages of the residues R are based on the sum of all residues R, i.e. on m. These new polyether polymers are a new type of material, which behaves very different to PEG in regard to the affinity towards anti-PEG antibodies (APAs) and have a low immunogenicity. They also have a low crystallinity up to amorphous structure. But the polyether polymers of the present invention behave very similar to PEG with respect to various features that are important for pharmaceutical applications, including aqueous solubility, hydration, cell viability and biocompatibility.

Key to the reduced interaction with APAs is the use of substituents on the PEG backbone. The increased spatial requirements of the said PEG copolymers impede or disable interaction with anti-PEG antibodies according to the specific “lock and key principle”. Besides the steric impact, the random or statistical distribution of the substituents over the polymer backbone and/or the random or statistical steric orientation of the substituents furthermore disable the development and formation of specific anti-polymer antibodies.

Key to the similarity of the features of PEG and the inventive polymers is the use of alkoxymethyl side chains. This maintains water solubility and other properties in view of PEG. A repeating group of the present polymer wherein R is a methoxymethyl group, i.e. (—CH₂—CH(—CH₂—OCH₃)—O)—, has the same molecular formula C₄H₈O₂ as two repeating units of PEG (—CH₂—CH₂—O)₂— and is therefore a constitutional isomer of two main chain repeating units of PEG. It comprises the same number of atoms and very similar functional groups. This leads to similar properties, especially to similar water solubility, which is important for applications in biological systems. The lack of reactive functional groups in the side chain like hydroxy, amino etc. reduces undesired interactions with biological systems.

Ethoxymethyl, n-propoxymethyl and iso-propoxymethyl may be used as residue R to adjust the properties of the polymers further, for example their solubility, interaction with lipids, properties in respect to the passage of biological membranes etc. However, these residues lead to a reduced solubility in water. The choice of such substituents also leads to a deviation from the behavior of PEG for other properties. For this reason, the amount of repeating units with the residues selected from the group consisting of ethoxymethyl, n-propoxymethyl and iso-propoxymethyl is limited to 50% of all residues R of the polymer of formula (I). Also in embodiments of the present invention in which at least 10% of residue R of the polymers is selected from the group consisting of ethoxymethyl, n-propoxymethyl and iso-propoxymethyl at least 10% are hydrogen and more preferred at least 25% are hydrogen. In a further preferred embodiment up to 20% of the residues R may be selected from the group consisting of ethoxymethyl, n-propoxymethyl and iso-propoxymethyl and even more preferred only up to 50% of the residues R may be selected from this group. These residues are not required for some of the uses of the polymers of the present invention in pharmaceutical applications. For this reason, polymers of the present invention are preferred where R is selected from the group consisting of hydrogen and methoxymethyl. Obviously, ethylene oxide repeating units in the inventive polymers also lead to similar properties with PEG. This selection therefore provides the polymers that are closest in behavior to PEG.

Higher homologues of methoxymethyl, i.e. where R is C4 alkoxymethyl and higher lead to increasingly low solubility in water and other detrimental properties and should therefore generally be avoided. They may however be used in small quantities when required, preferably in amounts of 10% or less and more preferably in amounts of 3% or less and most preferred 1% or less, based on the sum of all residues R selected from the group consisting of hydrogen, methoxymethyl, ethoxymethyl, n-propoxymethyl and iso-propoxymethyl; i.e. on m. If the polyether of the present invention comprises higher homologues of methoxymethyl, where R is C4-alkoxymethyl or higher, the same proviso for the presence of hydrogen applies with the same preferred amounts. Various other co-monomers may be also used in small amounts, i.e. preferably less than 3 wt %, more preferably less than 1 wt % and most preferred in less than 0.1 wt % based on the weight average molecular weight of the polymer of formula [I], if necessary. They may for example be used to introduce functional groups or side chains into the polymer. Best suited for the use in pharmaceutical application are however usually polymers of the present invention that comprise only the repeating units of formula (I).

As mentioned before, ethylene oxide repeating units and glycidyl methyl ether repeating units, i.e. wherein R is methoxymethyl, provide polymers with very similar properties. Because of the very similar properties, the ethylene oxide repeating units in the backbone of PEG may be completely substituted by methoxymethyl substituted repeating units, i.e. 100% of the residues R may be selected from methoxymethyl groups, which constitutes the homopolymer of glycidyl methyl ether. This is however not preferred. The properties of the polymers of the present invention are still closer to the properties of PEG, when at least some of the residues R are selected to be hydrogen. Furthermore, immunogenicity of copolymers will be reduced as compared to the homopolymer of glycidyl methyl ether, because they do not only differ in the orientation of side groups, but also side groups themselves. Preferred are therefore polymers for the present invention that are copolymers. The largest differences between the side groups and therefore the lowest immunogenicity is provided by polymers in which some of the repeating units have no side groups, i.e. where R is hydrogen.

This will ensure a lower immunogenicity. Preferred 10 to 90% of the residues R are hydrogen and 10 to 90% of the residues R are independently from each other selected from the group consisting of methoxymethyl, ethoxymethyl, n-propoxymethyl and iso-propoxymethyl. Even more preferred are 20 to 80% of the residues R are hydrogen and 20 to 80% of the residues R are independently from each other selected from the group consisting of methoxymethyl, ethoxymethyl, n-propoxymethyl and iso-propoxymethyl. The lowest immunogenicity provide polymers of the present invention, where about 50% of all ethylene oxide repeating units are substituted by alkyl glycidyl ethers. For this reason polymers are preferred where 30 to 70% of the residues R are hydrogen and 30 to 70% of the residues R are independently from each other selected from the group consisting of methoxymethyl, ethoxymethyl, n-propoxymethyl and iso-propoxymethyl and more preferred 40 to 60%. Especially useful in pharmaceutical applications is a polyether polymer of the present invention, wherein the polyether is a poly(glycidyl methyl ether-co-ethylene oxide) copolymer, i.e. wherein the residue R is selected from the group consisting of hydrogen and methoxymethyl. The same preferred ranges apply to embodiments where R is selected from the group consisting of hydrogen and methoxymethyl, i.e. wherein the polyether is a poly(glycidyl methyl ether-co-ethylene oxide) copolymer.

Especially preferred are polyether polymer of the present invention, wherein 30 to 70% of the residues R are hydrogen and the remaining residues R are methoxymethyl. These copolymers comprising repeating units where R is hydrogen combine most of the aforementioned advantageous properties. They have low immunogenicity, good water solubility, low crystallinity and are otherwise very similar to PEG.

A further aspect of the polymers of the present invention is that crystallinity, i.e. degree of crystallization, in comparison to PEG is reduced while retaining the same properties. Some of the polymers have amorphous structure depending on the specific composition. Statistical copolymers of EO with GME or other glycidyl ethers can systematically reduce or completely disable crystallization of PEG. The degree of crystallization of PEG and the related melting point (Table 5) plays a key role in numerous pharmaceutical applications, such as e.g., suppositories or solid dispersions of drugs. In other applications, crystallization is undesired, and fully amorphous PEG is required, for instance in highly hydrophilic and biocompatible polyurethane soft foams used among other purposes for wound dressing. Due to the random incorporation of side chains, the said copolymers of this invention permit to obtain fully amorphous PEG copolymer structures, if more than 25% of the glycidyl ether monomer is incorporated. This feature is relevant for biomaterials based on triblock and multiblock structures, for instance combining the polyether with biodegradable polylactide blocks.

m is in the range of 10 to 1000; m is preferably in the range of 20 to 900 and more preferably in the range of 30 to 800. Most preferably m is in the range of 30 to 700. These ranges correspond to the polymer weights of PEG that are most suited for medical applications and that are used in existing medical application for PEG. Furthermore, the polymers of the present invention have preferably a molecular weight Mn as determined by MALDI-TOF MS in the range of 500 to 50.000 g/mol, more preferably in the range of 1.000 to 50.000 g/mol and most preferably in the range of 1.000 to 30.000 g/mol. These ranges apply to all embodiments of the present invention.

Copolymers of the present invention may be random copolymers or statistical copolymers. Such copolymers provide the lowest immunogenicity, as they do not provide a blueprint for the immune system for antibodies. They are intrinsically resistant to an immune response and are therefore preferred embodiments of the present invention. These polymers may be prepared by a process of the present invention as discussed below.

In one embodiment of the present invention the polymers of the present invention may be block copolymers or may have a block like structure or a tapered or gradient structure. The methods to prepare such polymers are known to the expert in the art. In such embodiments it is preferred that no more than 5% of the macromolecules of the polymers comprise blocks with more than 15 ethylene oxide repeating units, more preferably that no more than 5% of the macromolecules of the polymers comprise blocks with more than 8 ethylene oxide repeating units. This reduces immunogenicity.

As described above, similarity to PEG is an important feature of the polymers of the present invention. This is especially true for the end groups of the polymer. Processing of PEG for the use in the pharmaceutical field, involves regularly the modification of the end groups. For this purpose, any end group known to be used for PEG can also be used for the present polymers. This includes not only the use of all known end groups for PEG, but also their modification including the kind of modification and the process of modification. If necessary, in order to allow the present polymers to function in this regard as similar or in fact identical to PEG, the present polymers may be provided with ethylene oxide repeating units on either end or both ends of the polymer, as depicted in Formula [Ia] to [Ic]. These are preferred embodiments of the present invention.

—(CH₂CH₂—O—)ₐ—(CH₂CHR—O—)ₘ— [Ia]

—(CH₂CHR—O—)ₘ—(CH₂CH₂—O—)ₐ— [Ib]

—(CH₂CH₂—O—)ₐ—(CH₂CHR—O—)ₘ—(CH₂CH₂—O—)ₐ— [Ic]

In these formulae the partial formulae —(CH₂CHR—O—)ₘ— depicts a polymer of the present invention as described herein and a may be any number, but is preferable a number in the range of 1 to 10 and more preferably in the range of 1 to 3. Polymers according to formulae [Ia] to [Ic] allow the modification of the termini of the present polymers exactly as PEG is modified in known processes and especially in present industrial processes. This allows for a smooth transition of established processes from PEG to the polymers of the present invention. Such can be prepared without much additional effort, as will be discussed below.

A further embodiment of the present invention, are polyether polymers represented by any of the following formula [II] and [IIa] to [IIc]:

X—(CH₂CHR—O—)ₘ—CH₂CHR—Y [II]

X—(CH₂CH₂—O—)ₐ—(CH₂CHR—O—)ₘ—CH₂CHR—Y [IIa]

X—(CH₂CHR—O—)ₘ—(CH₂CH₂—O—)ₐ—CH₂CH₂—Y [IIb]

X—(CH₂CH₂—O—)ₐ—(CH₂CHR—O—)ₘ—(CH₂CH₂—O—)ₐ—CH₂CH₂—Y [IIc]

wherein m is 19 to 999 and R is defined as in formula [I] and a is as defined in any of formulae [Ia] to [Ic]. X is the end group derived from the initiator of the reaction and the α-end group (alpha end group) and Y is the ω-end group (omega-end group).

X or Y or both may be chosen from any end group known for use in PEG. The same end groups may also be provided for the polymers of formulae [Ia] to [Ic].

X and Y may independently of each other or both comprise one or more functional group selected from the group consisting of acetal (dialkoxy), aldehyde (formyl), amide (carboxamido), azide, carbonate (alkoxycarbonyl)oxy), carboxyl (carboxy), carboxylic anhydride, ester (alkoxycarbonyl), ether, halo, haloformyl (carbonohaloridoyl), hemiacetal (alkoxyol), hemiketal (alkoxyol), hydroxy, imide (imido), imine (imino), ketal (dialkoxy), ketone (oyl), orthoester (trialkoxy), primary, secondary, tertiary amino group, primary, secondary and tertiary alkoxy group, sulfhydryl (sulfanyl, H—S—), thioether and combinations thereof. In a preferred embodiment X or Y may be selected from the group consisting of alkyl, hydrogen, hydroxy, alkoxy, sulfanyl, phthalimide, amide, amine and combinations thereof. X or Y or both may be a primary alkoxy group selected of the formulae R—CHₙ—O—, wherein R is linear, branched or cyclic alkyl or phenyl and n equals 1 to 20. Preferred are X or Y or both selected from the group consisting of methoxy, ethoxy, propoxy, butoxy, pentoxy, hexoxy, heptoxy, octoxy, nonoxy, decanoxy, 3-ethyl-butoxy, 2,3-dialkoxypropoxy and a combination thereof. X or Y or both may also be the alkoxy residue of an alcohol of the following formula:

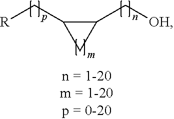

wherein R is hydrogen or linear, branched or cyclic alkyl or phenyl. X or Y or both may be the alkoxy residue of a secondary alcohol selected from any of the formulae

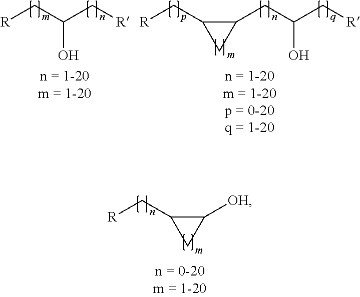

wherein R and R′ are independently from each other hydrogen or linear, branched or cyclic alkyl or phenyl. Among them a secondary alkoxy group is preferred that is selected from the group consisting of 2-propoxy, 2-butoxy, 2-pentoxy, 3-pentoxy, 2-hexoxy, 3-hexoxy, 2-heptoxy, 3-heptoxy, 4-heptoxy and cyclohexyloxy.

X or Y or both may also be selected from primary alkenyloxy groups, which may be linear, branched or cyclic. X or Y or both may also be the alkoxy residue of an alcohol of the following formula:

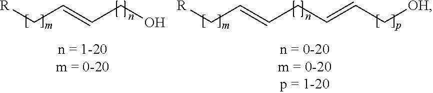

wherein R is hydrogen or linear, branched or cyclic alkyl or phenyl. Among them a primary alkenyloxy group is preferred that is selected from the group consisting of 2-propenoxy (allyloxy), 3-butenoxy, 2-butenoxy, 3-methyl-2-butenoxy, 4-pentenoxy, 3-pentenoxy, 2-pentenoxy and 3,5-hexadienoxy.

X or Y or both may further be selected from secondary alkenyloxy groups, which may be branched or cyclic. X or Y or both may be the alkoxy residue of any of the following formulae:

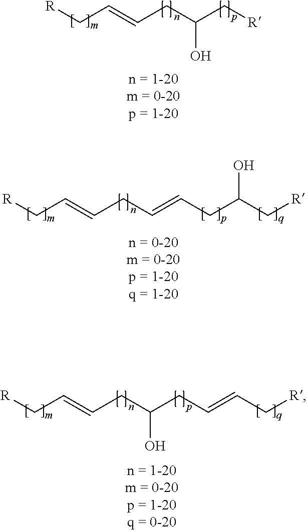

wherein R and R′ are independently from each other hydrogen or linear, branched or cyclic alkyl or phenyl. The secondary alkenyloxy group may be of any of the formulae 4-penten-2-oxy, 3-penten-2-oxy, 5-hexen-2-oxy, 4-hexen-2-oxy, 3-hexen-2-oxy, 4,6-heptadien-2-oxy, 1,4-pentadien-3-oxy.

X or Y or both may further be selected from aryloxy and heteroaryloxy groups. Benzyloxy groups are especially preferred. X or Y or both may be the aryloxy or benzyloxy residue of an alcohol of any of the following formulae:

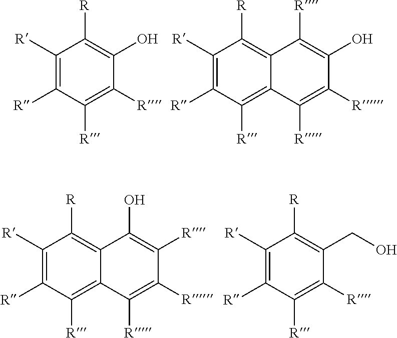

wherein R, R″, R′″, R″″ R′″″ and R″″″ are independently from each other hydrogen or linear, branched or cyclic alkyl or phenyl. X or Y or both are preferably selected from the group consisting of benzyloxy, phenyloxy and naphthyloxy.

X or Y or both may be multifunctional alkoxy groups, i.e. may have multiple ether bonds to the α- or ω-position of a plurality of macromolecules of the polymers of the present invention according to formulae [II] or [IIa] to [IIc]. Any suitable polyol may be selected therefore. X or Y or both may especially be selected from the group consisting of polyethers of carbohydrates, especially of Ribose, polyethers of 1,1,1-trimethylolpropane, polyethers of glycerol, polyethers of saccharides or hydrogenated saccharides.

Further, Y may be selected from the group consisting of acrylamide, acrylates, aldehyde, alkyne, amine, aminooxy, azide, benzotriazole carbonate, carboxylic acid, chloroformate, cyanuric chloride, dithioester, epoxide, fluorescein, hydrazide, imidazoyl formate, iminoester, isocyanate, maleimide, mesylate, methacrylate, NHS ester, nitrobenzoate, nitrophenylcarbonate, succinimidyl active ester, succinimidyl carbonate, N-succinimidyl carbonate, succinimidyl succinate, thiocarbonate, thiol, thiol carboxylic acid, tosylate, triflate, vinyl sulfone and xanthate.

It is especially preferred that X is selected from the group consisting of hydrogen, alkyl, hydroxy, sulfanyl, C1-C10-alkoxy, C1-C10-thioalkoxy, amino, amino-C1-C10-alkoxy, dibenzylamino-C1-C10-alkoxy, amide, N-heterocyclic carbenes and N-heterocyclic olefins. It is also especially preferred that Y is selected from the group consisting of hydrogen, hydroxy, alkoxy, —CH₂—C(═O)—R wherein R is as defined above, —CHO, alkylcarbonyloxy, alkyoxycarbonyloxy, amine, alkylamine, carboxamido, azide, halogen, sulfanyl, thioalkoxy and sulfonate. In addition, for any alkoxy rest defined herein for the residue X the corresponding thioalkoxy residue may be used, i.e. where a sulfur atom is present instead of the oxygen atom.

Most preferred are polymers of the present invention, where X and/or Y are selected from the group consisting of low molecular weight drugs, nanocarriers, liposomal structures, peptides, polypeptides, glycoproteins, polynucleotide, polysaccharides, lipid structures, liposomes, surfaces and interfaces, which are bonded directly by a covalent bond or by a spacer to the polymer. Nanocarriers usable in the present invention are for example selected from the group consisting of carbon nanotubes (CNT), dendrimers, gold nanorods, lipid nanoparticels, liposomes, micelles, nanocrystals, niosomes, polymeric nanoparticles (PNP), solid lipid nanoparticles (SLNs) and virus-based nanoparticles (VNP).

The polymers of the present invention have dispersities of 1.15 or less. The dispersities of the present invention were determined using size exclusion chromatography (SEC) in DMF by calibration with PEG standards as described in more detail in the examples. They are suitable for applications where low dispersities are required, as for example in pharmaceutical, medical and biological applications. Dispersity of polymers in pharmaceutical, medical and biological applications is of great importance, as macromolecules of different molecular weight may behave differently in biological systems. They may for example differ in solubility in water, membrane permeability, crystallinity and renal clearance. Since a uniform behavior of all molecules is desired in medical applications, a low dispersity is often required. Most preferred are therefore polymers of the present invention that have a dispersity of 1.10 or less. This applies to all embodiments of the present invention. A dispersity of 1.10 or less is required for most medical and pharmaceutical applications. Most preferred are polymers of the present invention which have a dispersity that is lower than 1.08.

Preferred embodiments of the present invention are polyether polymers represented by the following formula [II]:

X—(CH₂CHR—O—)ₘCH₂CHR—Y [II]

wherein residues R are independently from each other selected from the group consisting of hydrogen, methoxymethyl, ethoxymethyl, n-propoxymethyl and iso-propoxymethyl; 1 to 100% of the residues R are methoxymethyl; up to 50% of the residues R may be selected from the group consisting of ethoxymethyl, n-propoxymethyl and iso-propoxymethyl; with the proviso that at least one residue R is hydrogen, if at least one residue R is selected from the group consisting of ethoxymethyl, n-propoxymethyl and iso-propoxymethyl; X is selected from the group consisting of hydrogen, alkyl, hydroxy, sulfanyl, C1-C10-alkoxy, C1-C10-thioalkoxy, amino, amino-C1-C10-alkoxy, dibenzylamino-C1-C10-alkoxy, amide, N-heterocyclic carbenes and N-heterocyclic olefins; Y is selected from the group consisting of hydrogen, hydroxy, alkoxy, —CH₂—C(═O)—R wherein R is as defined above, —CHO, alkylcarbonyloxy, alkyoxycarbonyloxy, amine, alkylamine, carboxamido, azide, halogen, sulfanyl, thioalkoxy, N-succinimidyl carbonate and sulfonate; X and/or Y may also be selected from the group consisting of low molecular weight drugs, nanocarriers, liposomal structures, peptides, polypeptides, glycoproteins, polynucleotides, polysaccharides, lipid structures, liposomes, surfaces and interfaces, which are bonded directly by a covalent bond or by a spacer to the polymer, m is in the range of from 19 to 999, characterized in that the dispersity is 1.15 or less. Especially preferred is N-succinimidyl carbonate.

An especially preferred embodiment of the present invention is a polyether polymer of the present invention, wherein X is alkoxy and Y is hydroxy. Further preferably X is a C1-C10-alkoxy group and Y is hydroxy and even more preferably X is a C1-C3 linear alkoxy group and Y is hydroxy. These embodiments are analogous to commercially available PEG derivates. Most preferred X is methoxy and Y is OH, which is analogous to mPEG.

As discussed in regard to the dispersity, a unified behavior of all molecules in drugs and excipients used in pharmaceutical and medical applications is preferred. The end-group fidelity of the polymers of the present invention may be determined by MALDI TOF MS or by a combination of MALDI TOF MS with ¹H NMR by known methods. The polymers of the present invention have preferably an end-group fidelity of at least 95% in regard to the group X and more preferably of at least 98%. In a further embodiment the polymers of the present invention have preferably an end-group fidelity of at least 95% in regard to the group Y and more preferably of at least 98%. Even more preferred are polyether polymers of the present invention, wherein the end-group fidelity of the polymer in regard to group X and group Y is at least 95% and most preferably of at least 98%. Especially preferred are polyether polymers of the present invention, wherein the end-group fidelity of the polymer in regard to group X and group Y is at least 95% and the dispersity is 1.10 or less and most preferred wherein the end-group fidelity of the polymer in regard to group X and group Y is at least 98% and the dispersity is 1.10 or less.

In another embodiment of the present invention the polyether polymers are polyether polymers represented by the following formula [II]:

X—(CH₂CHR—O—)ₘCH₂CHR—Y [II]

wherein R has the same meaning as mentioned in the other embodiments of [I] and [II], wherein X is selected from group X as mentioned in the other embodiments of [I] and [II] or from the group consisting of hydrogen or X′,
wherein X′ is selected from the group consisting of

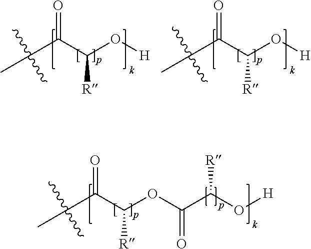

wherein R″═H or alkyl, alkenyl, aryl, alkoxymethyl, alkenyloxymethyl, aryloxymethyl, aminomethyl and thioalkoxymethyl, preferably R″ is alkyl, i.e.

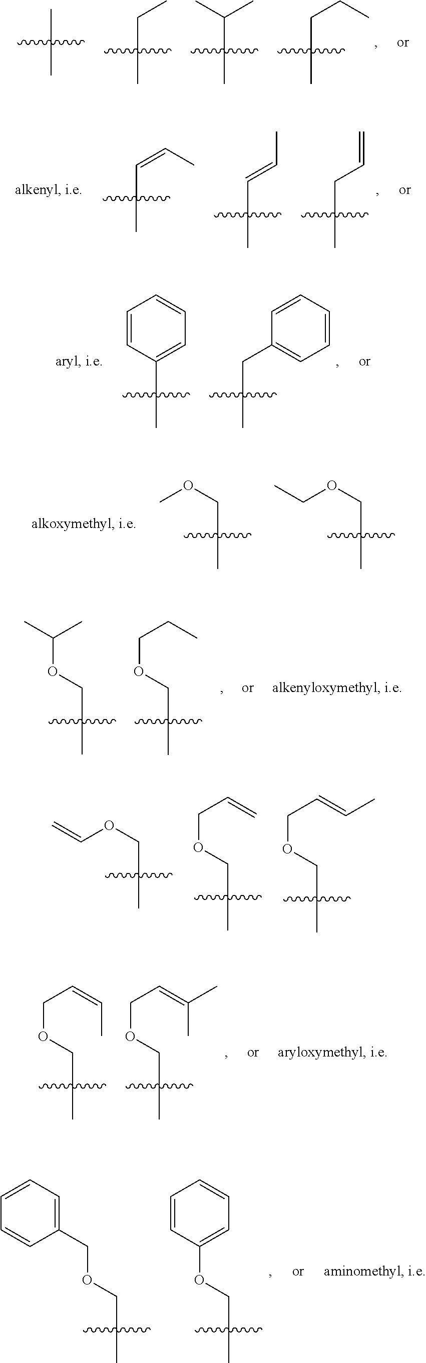

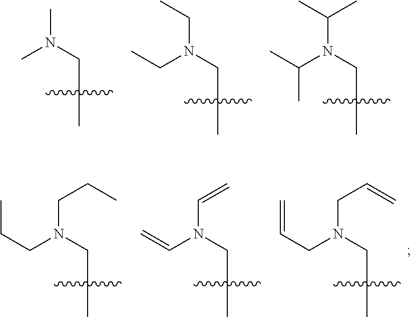

wherein
p=1, 2, 3, 4 or 5, and
k=5-500, preferably 5-250, most preferably 10-200; and

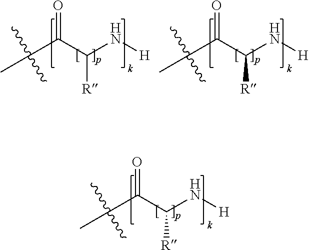

wherein R″═H or alkyl, alkenyl, aryl, alkoxymethyl, alkenyloxymethyl, aryloxymethyl, aminomethyl and thioalkoxymethyl, preferably R″ is alkyl, i.e.

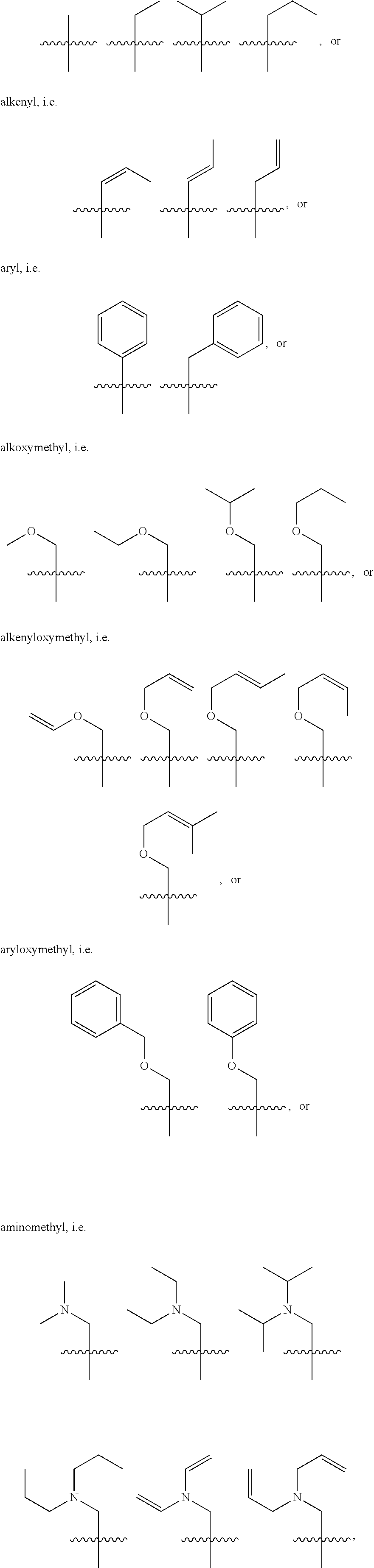

wherein
p=1, 2, 3, 4 or 5 and
k=5-500; preferably 5-250, most preferably 10-200; and

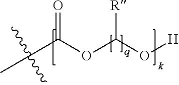

wherein R″═H or alkyl, alkenyl, aryl, alkoxymethyl, alkenyloxymethyl, aryloxymethyl, aminomethyl and thioalkoxymethyl, preferably

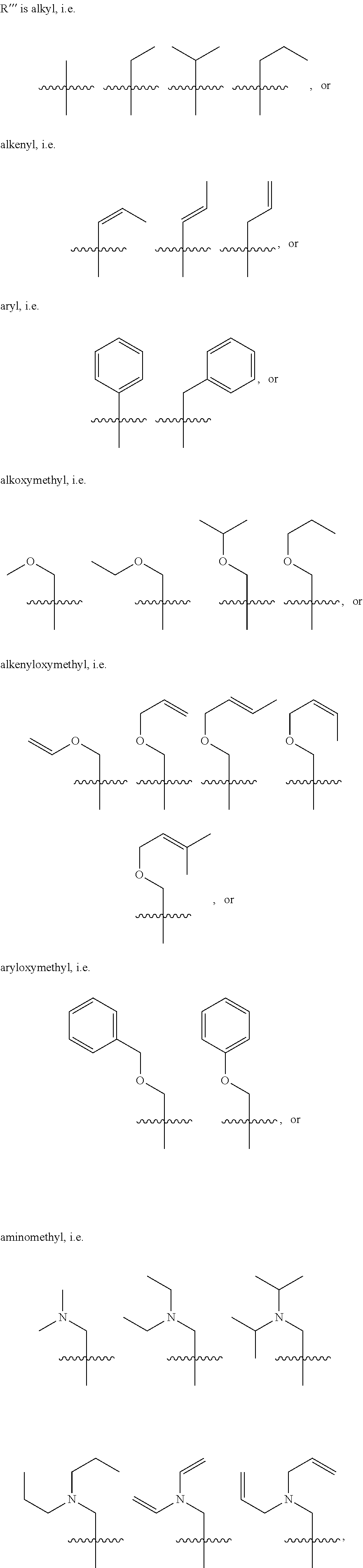

wherein
q=3, 4 or 5 and
k=5-500, preferably 5-250, most preferably 10-200;
and

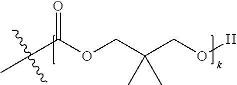

wherein k=5-500, preferably 5-250, most preferably 10-200; and
wherein Y is Z—W meaning composed of substituent Z and W linked via a covalent bond, wherein
Z is —O—R′—O— and
W is —R—CHCH₂—(O—CHRCH2—)ₘ—V;
wherein V is X or X′;
wherein R′ is selected from the group consisting of

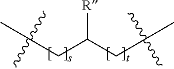

wherein
R″═H or alkyl, alkenyl, aryl, alkoxymethyl, alkenyloxymethyl, aryloxymethyl, aminomethyl and thioalkoxymethyl, preferably
R″ is alkyl, i.e.

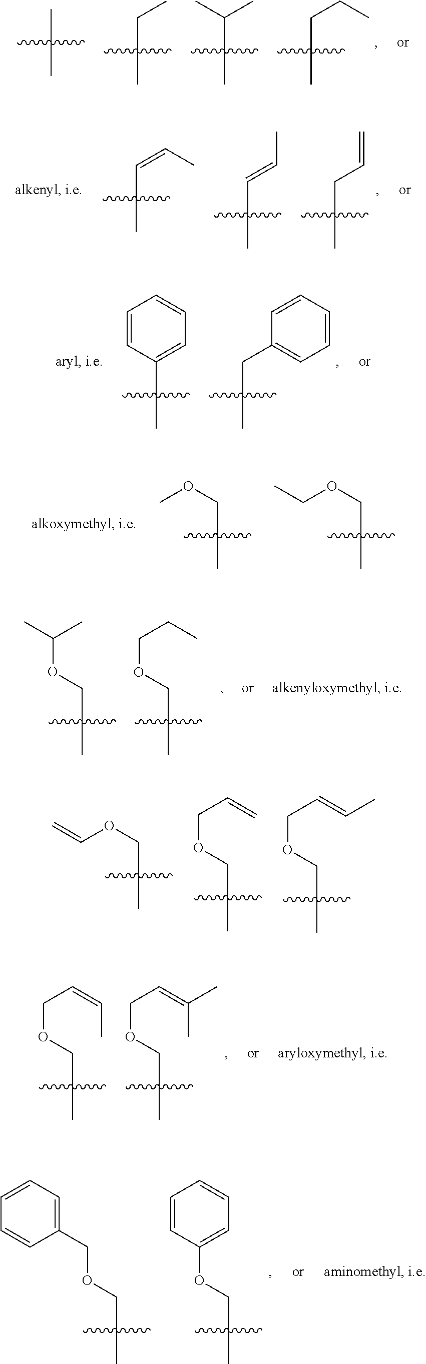

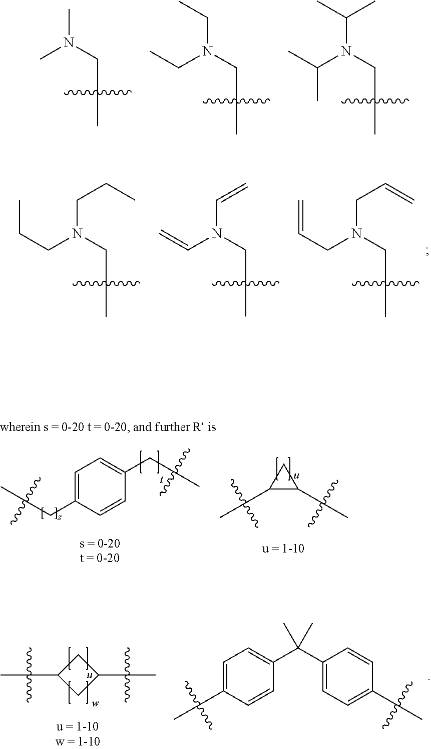

In another embodiment block copolymers of P(EG-co-GME) and poly(L-lactid acid) (PLLA) are preferred having —O—CH3, —O—Bz, or —OH as functional group at the end of the polymer chain, i.e. diblock polymers MeO—P(EG-co-GME)-b-PLLA-OH, BzO-P(EG-co-GME)-b-PLLA-OH or HO—P(EG-co-GME)-b-PLLA-OH and HO—P(EG-co-GME)-b-PLLA-OH and triblock polymers such as HO—PLLA-b-P(EG-co-GME)-b-PLLA-OH.

In all embodiments of the invention m is in the range of from 9 to 999, more preferably in the range of 30 to 800. Most preferably m is in the range of 30 to 700. These ranges correspond to the polymer weights of PEG that are most suited for medical applications and that are used in existing medical application for PEG. Furthermore, the polymers of the present invention have preferably a molecular weight Mn as determined by MALDI-TOF in the range of 500 to 50.000 g/mol, more preferably in the range of 1.000 to 50.000 g/mol and most preferably in the range of 1.000 to 30.000 g/mol. These ranges apply to all embodiments of the present invention.

The dispersity of all compounds of the present invention is 1.15 or less measured by size exclusion chromatography in DMF by calibration with PEG standards. The end-group fidelity of the polymer in regard to group X and group Y is at least 95% and the dispersity is 1.10 or less and most preferred wherein the end-group fidelity of the polymer in regard to group X and group Y is at least 98% and the dispersity is 1.10 or less.

As aforementioned, the polymers of the present invention have similar properties as PEG, especially in biological systems. The present polymers may therefore substitute PEG in most of his applications. The polymers of the present invention are especially designed for use in the pharmaceutical and medical field. They may also be used with the same benefits in veterinary medicine and in other biological applications. In these fields the polymers of the present invention can fulfill the same functions as PEG but have reduced immunogenicity and antigenicity. Because of their irregular structure, they do not elicit a strong immune response. The inventive polymers show especially a low response to anti-PEG antibodies, as evidenced by ELISA tests. Conjugates used for masking drugs and other pharmaceutically active compounds usually comprise PEG in addition to a physiologically active compound or an adjuvant. The polymers of the present invention may be used instead of PEG in any such application. At the same time the immune response elicited by the present polymers will be weak or non-existent thereby resolving one of the main problems in the use of PEG.

A further embodiment of the present invention is therefore a conjugate comprising a polyether polymer of any of the preceding claims and a substrate. The conjugates of the present invention may in the following also be named polyether-conjugated substrates or polymer-conjugated substrates. In the conjugates of the present invention a polymer of the present invention is bonded to a substrate directly or indirectly by a covalent bond. The polymer of the present invention may be bonded to the substrate by a spacer, which constitutes an indirect bond. The substrate may be selected from the group consisting of a pharmaceutically active compound, an adjuvant, a surface and interfaces. The pharmaceutically active compound is preferably selected from the group consisting of low molecular weight drugs, peptides, polypeptides, proteins, glycoproteins, polynucleotides and polysaccharides. The adjuvant is preferably a vesicle or carrier, which are preferably used to carry a pharmaceutically active compound. The adjuvant is preferably selected from the group consisting of nanocarriers, liposomal structures, lipid structures and liposomes. Nanocarriers usable in the present invention are for example selected from the group consisting of carbon nanotubes (CNT), dendrimers, gold nanorods, lipid nanoparticles, liposomes, micelles, nanocrystals, niosomes, polymeric nanoparticles (PNP), solid lipid nanoparticles (SLNs) and virus-based nanoparticles (VNP). In a further preferred embodiment the substrate is selected from the group consisting of low molecular weight drugs, nanocarriers, liposomal structures, peptides, polypeptides, proteins, glycoproteins, polynucleotides, polysaccharides, lipid structures, liposomes, surfaces and interfaces. Also in these embodiments the substrate may be bonded directly by a covalent bond or by a spacer to the polymer. Especially preferred are conjugates of the present invention with bovine serum albumin.

The polymer of the present invention may be bonded to the substrate directly by a covalent bond or by a spacer or any kind. Preferably a hydroxy-residue at the ω-terminal of the polymer of the present invention is used to bond a substrate or a spacer to the polymer. This is most economical, as the polymers of the present invention where X is an inert group and Y is OH can be most easily be prepared (see Examples). The conjugation of a substrate to the OH group at the ω-terminal end of PEG is well established. The same processes may be used for the polymers of the present invention. The substrate may however also be bonded to the α-terminal of the polymer. The polymers of the present invention can also be bonded to more than one substrate. This can be done by use of the α-terminal and the ω-terminal of the polymer and by using functional end groups of the polymer or spacer that can bond to a plurality of substrates. This may also be achieved by use of co-monomers with functional groups to which a substrate may be bonded. The bond between the polymers of the present invention and the substrate may be prepared by any means currently used for bonding PEG to substrates. It is also possible to bond a plurality of macromolecules of the polymers of the present invention to one substrate. This is especially useful for vesicle of all kinds including liposomes.

Typical examples for PEG conjugates are the lipid nanoparticles that are essential for vaccines against Covid-19 of the firms BioNTech SE and Pfizer Inc. and Moderna. It comprises a PEG conjugate of lipids, and the lipid nanoparticles (LNPs) are used to encapsulate and protect mRNA, which is the pharmaceutically active substance. The PEG in these conjugates may be substituted by the polymers of the present invention, thereby retaining the effects of the PEG polymer in the conjugate and at the same time reducing the immune response to the conjugate. A further vaccine of this kind is the Covid-19 vaccine of Curevac. Further drugs that comprise PEG conjugates wherein the PEG could be substituted by the present polymers are listed in the following.

TABLE 1

| Trade names | Chemical Structure | Indication |
|---|---|---|
| Jivi ® | 60k-PEG recombinant Factor VII | Hemophilia A |
| | antihemophilic factor | |
| Palynziq ® | 2K-PEG-rhu-Phenylalanine | Phenyl-ketonuria |
| | ammonia-lyase, Pegvaliase-pqpz | |
| Adynovate ® | 20K-PEG-Factor VIII | Hemophilia A |
| | Antihemophilic Factor VIII | |
| Revolixys ® kit | 40K-PEG-RNA aptamer + reverse | Anti-coagulation |
| | agent, Factor-IXa blocker, | |
| | Pegnivacogin /Anivamersen | |
| Onicyde ® | 2K-PEG-Liposomal irinotecan | Metastatic pancreatic cancer |
| | hydrochloride trihydrate | |
| Plegridy ® | 20k PEG-Interferon β-1a | Relapsing from multiple |
| | | sclerosis |
| Movantic ® | <1k PEG-Naloxegol | Opioid-induced constipation |
| Omontys ® | 40K-PEG-Erythropoietin-mimetic | Anemia associated with |
| | peptide, Peginesatide | chronic kidney disease |
| Sylatron ® | 12K-PEG-Interferon α-2b | Melanoma |
| Krystexxa ® | 10k-PEG-Uricase, Pegloticase | Gout |
| Cimzia ® | 40k-PEG-Certolizumab | Rheumatoid arthritis, Crohn's |
| | | disease, Axial |
| | | spondyloarthritis and psoriatic |
| | | arthritis |
| Mircera ® | 30K-PEG- erythropoietin | Anemia associated with |
| | (epoetin) β | chronic kidney disease |
| Macugen ® | 40K-PEG-anti-VEGF aptamer, | Age-related macular |
| | Pegaptanib | degeneration |
| Somavert ® | 5K-PEG-rhuGH (human growth | Acromegaly |
| | hormone), Pegvisomant | |
| Neulasta ® | 20K-PEG-Granulocyte colony | Neutropenia |
| | stimulating factor, Pegfilgrastim | |
| Pegasys ® | 40K-PEG-interferon α-2 | Hepatitis C und B |
| PegIntron ® | 12K-PEG-interferon α-2b | Hepatitis C und B |
| Doxil ®/Caelyx ® | 2K-PEG-Liposomal doxorubicin HCl | Cancer |
| Oncaspar ® | 5K-PEGylated L-asparaginase, | Acute lymphoblastic leukemia |
| | Pegaspargase | |
| Adagen ® | 5K-PEG-adenosine deaminase | Severe combined |
| | (bovine), Pegademase | immunodeficiency disease |
| | | (SCID) |
| Irinotecan ® | PEGylated liposomal irinotecan | Metastatic pancreatic cancer |
| | hydrochloride trihydrate | |
| Lonquez ® | 20k PEG-rhG-CSF | Neutropenia |
| Jintrolong ®¹ | Branched 40k PEG hGH | hGH deficiency |
| Neulapeg ® | 20k-PEG rhG-CSF mutein | Neutropenia |
| Rebinyn ®/ | Branched 40k PEG Recombinant | Hemophilia B |
| Refixia ® | factor VIII | |
| Asparlas ® | 5k-PEG L-Asparaginase | Acture lymphoblastic leukemia |
| Revcovi ® | 5k-PEG Recombinant B-domain | Hemophilia A |
| | truncated factor VIII | |
| Besremi ®³ | Branched 40k PEG Proline- | Polycythemia vera |
| | interferon α-2b | |
| PEGPH20 ®³ | 30k-PEG recombinant human | Pancreatic cancer |
| | hyaluronidase | |
| Pegargiminase | 20k PEG Arginine deiminase | Hepatocellular carcinoma |
| | Interleukin-2 | |
| NKTR-214 | Branched PEG-20k Fmoc- | Melanoma, rencal cell |
| | succinimidyl carbonate | carcinoma |
| PRX-201³ | Bifunctional PEG2k α- | Fabry disease |
| | Galactosidase A | |
| Peginterferon | 20k PEG recombinant interferon | Hepatitis C and B², Hepatitis |
| lambda-1a | lambda-1a | D³ |
| Abicipar pegol | 20k PEG anti-VEGF DARPin | Neovascular age related |
| | | macular degeneration |
| TransCon GH ®³ | Four-arm-40PEG spacer hGH | Growth hormone deficiency |
| AM0010² | 30k PEG-Interleukin-10 | Metastatic pancreatic cancer |
| Rolontis ®³ | Bifunctional PEG rhG-CSF | Neutropenia |
| Efpeglenatide³ | Bifunctional PEG Exendin-4 | Type-2 diabetes mellitus |
| | (glucagon-like peptide-1 receptor | |
| | agonist) | |
| Pegbelfermin² | 30k PEG fibroblast growth factor- | Fibrosis, liver disorders, |
| | 21 mutein | nonalcoholic steatohepatitis, |
| | | type 2 diabetes mellitus |
| Imrestor ® | 20k PEG recombinant bovine G- | Reducing risk of clinical |
| (veterinary use) | CSF mutein | mastitis |
| Pegfilgrastim biosimilars: | 20K-PEG-Granulocyte colony | Neutropenia |
| Fulphila (Mylan ®) | stimulating factor, Pegfilgrastim, | |
| Lapelga/Pelgraz | rhG-CSF | |
| (Apobiologix ®), | | |
| Udenyca | | |
| (Coherus Biosciences ®), | | |
| Pelmeg (Mundipharma ®), | | |
| Ziextenzo (Sandoz ®) | | |
| Esperoct ® | Branched-PEG40k-SA-CMP, | Hemophilia A |
| | enzymatic glycopegylation at | |
| | O-glycan on Ser-750 | |

¹only in China
²Phase II
³Phase III

In another embodiment of the invention the invention relates to a process for the preparation of a polyether polymer by anionic ring-opening copolymerization (AROP) comprising the steps of:
Providing an anion An⁻,
Adding at least one monomer of the formula

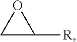

wherein R is as defined as described for any of formulae [I] or [II],
Allowing the polymerization to proceed at a temperature in the range of −10 to 90° C.,
   wherein the monomer comprises less than 1 wt % of epichlorohydrin.

In the process of the present invention the anion An must be an anion suitable for anionic ring-opening copolymerization (AROP). The expert in the art can easily choose among the known anions for this purpose. The An anion may be selected from the groups consisting of alkyl anion, hydride, OH⁻, alkoxy⁻, SH⁻, thioalkoxy⁻, amine anion, amide anion, imide anion and combinations thereof. Alkyl anions and hydride anions may be provided in the form of metal alkyl or metal hydride compounds. The alkoxy anion and the thioalkoxy anion may be the corresponding anion with the same structure as any of the alkoxy groups and aryloxy groups and aralkoxy groups defined herein for the residue X. Preferably however the alkoxy anion and the thioalkoxy anion are not tertiary alkoxy anions. The imide anion is preferably a phthalimide anion. Polymers of the Formulae [Ia], [Ic], [IIa] and [IIc] may be prepared by first adding the corresponding amount of ethylene oxide to the anion An and allowing the polymerization to proceed at a temperature in the range of −10 to 90° C. until all of the ethylene oxide is consumed. Subsequently the steps of adding least one monomer of the formula

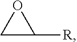

and allowing the polymerization to proceed at a temperature in the range of −10 to 90° C. are performed. In this manner the repeating units adjacent to the α-terminal of the polymers of the present invention are provided with exactly the same structure as PEG. Polymers of the Formulae [Ia], [Ic], [IIa] and [IIc] may also be prepared by use of anions of the formulae X—(CH₂CH₂—O—)ₐ⁻, wherein a is as defined for any of the Formulae [Ia], [Ic], [IIa] and [IIc] and X is any residue as defined herein for X. X′ is any residue as defined herein for X′. This is the preferred method. Especially preferred is the use of such anions of the formulae X—(CH₂CH₂—O—)ₐ⁻, in which X is alkyl, alkoxy, dialkylamin anion or (RCO)₂N⁻. Even more preferred is the use of anions selected from the group consisting of MeOCH₂CH₂O⁻, MeO(CH₂CH₂O)₂⁻, BenzylOCH₂CH₂O⁻, BzO(CH₂CH₂O)₂⁻, (Bz)₂N—CH₂CH₂O⁻, (Bz)₂N—(CH₂CH₂O)₂⁻, phthalimide-CH₂CH₂O⁻, phthalimide-(CH₂CH₂O)₂⁻, wherein Me is methyl and Bz is benzyl. Most preferred are MeO(CH₂CH₂O)₂⁻, BzOCH₂CH₂O and (Bz)₂N—CH₂CH₂o⁻.

In addition, in order to synthesize bifunctional initiated α,ω-OH—P(EG-co-GME) copolymers that enables the synthesis of ABA triblock copolymers, P(EG-co-GME) bearing two hydroxyl-termini is used as an macroinitiator for ring-opening polymerization of cyclic polyesters, cyclic diesters or cyclic monoesters, e.g. L-lactide, D-lactide, meso-lactide, cyclic carbonates, such as trimethylene carbonate (TMC), 2,2-dimethyl-trimethylene carbonate (DTC), 5-butyl-1,3-dioxan-2-one and 1,3-dioxepan-2-one; glycolide, other cyclic esters and lactams, N-carboxy anhydrides. In an embodiment of the invention the reaction is performed using L-lactide enabling the synthesis of e.g. PLLA-b-P(EG-co-GME)-b-PLLA, wherein PLLA means

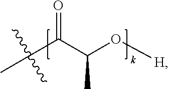

wherein k=5-500, preferably 5-250, most preferably 10-200.

The polymerization is catalyzed by Lewis acids e.g. triethylaluminum (AlEt₃) and tin(II) bis-(2-ethylhexanoate) (Sn(Oct)2) or organic bases e.g., DBU (1,8-Diazabicyclo[5.4.0]undec-7-ene) and N-methyl-1,5,7-triazabicyclo[4.4.0]dec-5-ene. The described procedure is not limited to triblock copolymers. With the utilization of a monofunctional P(EG-co-GME) macroinitiator, diblock copolymers (P(EG-co-GME)-b-PLLA) are obtained.

In these embodiments An⁻ can be selected from the group consisting of alkoxy⁻, thioalkoxy⁻ and amine anion.

Further, in a final step of the preparation, addition of a small amount of pure EO is possible to ensure primary hydroxyl end groups for the ω-terminal of the polymer that can undergo all established coupling strategies to therapeutic entities. This leads to polymers of the present invention with Formulae [Ib], [Ic], [IIb] and [IIc].

The counter ion to the An⁻ anion is preferably selected from the group consisting of Na⁺, K⁺ and Cs⁺. The anion An- may be provided in an inert solvent.

The anionic ring-opening polymerization of epoxides can be conducted in non-protic solvents like DMSO or other polar aprotic solvents, e.g., tetrahydrofuran (THF), methyl-THF, cyclopentyl methyl ether (CPME), benzene, toluene, xylene, HMPA, dioxane, diglyme etc. Surprisingly, it was found that random copolymers can be synthesized by using the non-protic solvent DMSO and statistical copolymers can be synthesized by using polar aprotic solvents e.g. toluene. This can be seen from FIGS. 9, 10, 14 and 15.

The solvent is preferably a non-protic solvent and most preferably dimethyl sulfoxide (DMSO). Also preferably the reaction is performed in the same solvent.

Anionic ring-opening polymerizations with alkyl glycidyl ethers are more prone to undergo chain transfer reactions as follows:

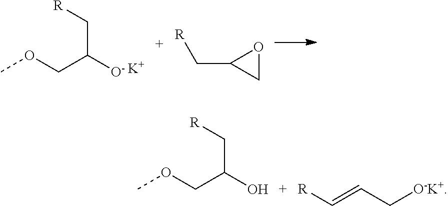

These side reactions increase with the temperature. Therefore, high molecular weight polymers can only be obtained, if the reaction temperature is not above 90° C. The chain transfer reaction results in a broad distribution of molecular weight and therefore in a high dispersity. Therefore, lower polymerization temperatures also provide polymers of the present invention with lower dispersity. On the other hand, alkyl glycidyl ethers are as reactive in the anionic ring-opening polymerization as ethylene oxide. For this reason the polymerization may be performed at temperatures as low as −10° C. Preferably the polymerization is performed at a temperature in the range of 10 to 70° C. and more preferred at a temperature in the range of 10 to 60° C.

A further problem in anionic ring-opening polymerizations is that commercially available alkyl glycidyl ethers are prepared from epichlorohydrin. Part of the epichlorohydrin remains in the alkyl glycidyl ether. Presence of alkyl glycidyl ethers leads to termination reactions as follows:

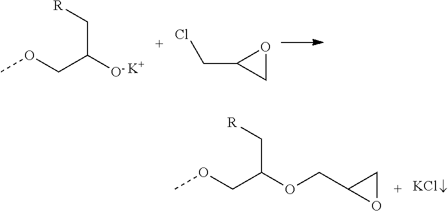

This in turn leads to low molecular weights and high dispersity of the product. The polymers of the present invention are therefore only accessible by reaction temperatures of −10 to 90° C. or less and by the use of alkyl glycidyl ethers that comprise less than 1 wt % of epichlorohydrin. Preferably the alkyl glycidyl ethers comprise less than 0.5 wt % of epichlorohydrin and more preferably less than 0.1 wt %. Most preferably the alkyl glycidyl ether are free of epichlorohydrin. Removal of epichlorohydrin from the glycidyl methyl ether to the extent necessary is impossible, because the glycidyl methyl ether and epichlorohydrin have similar boiling points, e.g. epichlorohydrin 117.9° C., glycidyl methyl ether 110° C. Procedures for the preparation of epichlorohydrin free alkyl glycidyl ethers is provided in Example 1 of the present invention.

Furthermore, high temperatures also lead to side reactions of the starting anion An⁻ or the group X of the living polymer. Limitation of the temperature in the aforementioned range therefore also improves the end-group fidelity at the α-terminal of the polymers of the present invention and allows for end-group fidelities of 95% or more of the X residue. Other known methods for the polymerization of the alkyl glycidyl ethers do not provide the low dispersity required for the present polymers. They also do not allow for the same high end-group fidelity.

In order to provide random or statistical copolymers or the present invention, it is necessary to add at least two different kinds of monomers to the anion. The reactivity ratio for both alkyl glycidyl ether employed and ethylene oxide is in the range of 0.7 to 1.3 or 0.6 to 1.7. For glycidyl methyl ether and ethylene oxide the reactivity ratio is in the range of 0.98 to 1.02 in polymerizations in DMSO. This results in almost ideally random copolymers. Other forms of copolymers i.e. with a block structure, block like structure or a tapered or gradient structure can be prepared according to known methods. Block copolymers can easily be prepared by subsequent additions of different monomers to the living polymer. Therefore, a process of the present invention is preferred, wherein the step of adding at least one monomer and the step of allowing the polymerization to proceed are repeated at least one time, whereby at least one different monomer is used than the first time.

A further embodiment of the present invention is the use of the polyether polymers of the present invention for the preparation of a conjugate of the polymers with a substrate. The substrate is as defined herein for the conjugates of the present invention. The substrate is preferably a bioactive compound. There is a well-developed chemistry for the preparation of conjugates of PEG, which is known to the expert in the art. The same chemistry may be used for the preparation of a conjugate of the present invention. Preferred is a use of the present invention, for the preparation of conjugated lipids for use in vaccines, in particular based on lipid nanoparticles. These nanoparticles are preferably nanoparticles, as used against COVID-19. Preferred are conjugates of polymers of the present invention with bovine serum albumin (BSA). Especially preferred are conjugates with polymers of the present invention where the y group of the polymer is a N-succinimidyl carbonate group. Even more preferred are conjugates of the present polymer where bovine serum albumin in conjugates by means of such a group Y to a polymer of the present invention. Conjugation of α-BzO-ω-N-succinimidyl carbonate-P(EG-co-GME) to bovine serum albumin if most preferred.

An additional embodiment for the present invention concerns the preparation of conjugates of the present invention. As discussed above, the conjugates may be prepared by any known process for the preparation of conjugates of PEG. The present invention provides additionally a process, where firstly the process for the preparation of the polymers of the present invention is performed and then a substrate is added to the living polymer, wherein the substrate has a functional group that reacts with the living polymer to form a conjugate.

### BRIEF DESCRIPTION OF THE FIGURES:

FIG. 1: FIG. 1 shows the results of ELISA test of three of the polymers of the present invention and mPEG.

FIGS. 2 to 4: These figures show the SEC traces of polymers of the present invention synthesized in DMSO and FIG. 4 shows in addition the SEC trace of commercially available mPEG.

FIG. 5: FIG. 5 shows the ¹H NMR spectrum of α-BzO-P(EG_{0.51}-co-GME_{0.49}).

FIG. 6: FIG. 6 shows the MALDI-TOF spectrum of α-BzO-P(EG_{0.51}-co-GME_{0.49}).

FIG. 7: FIG. 7 shows cloud point of α-BzO-P(EG_{0.51}-co-GME_{0.49}).

FIG. 8: FIG. 8 shows the MALDI-TOF spectrum of α-MeO—P(PEG_{0.88}-b-PGME_{0.22}), i.e. a block copolymer of the present invention.

FIG. 9: FIG. 9 shows the decreasing ¹H NMR signals of monomers during the preparation of a polymer of the present invention.

FIG. 10: FIG. 10 shows the result of a study of the relative reactivity of ethylene oxide and glycidyl methyl ether in the anionic ring opening polymerization.

FIG. 11: FIG. 11 shows the mechanism and side reactions of monomer-activated ring-opening polymerization using triisobutylaluminium leading to low end-group fidelity, wherein a) shows the epoxide activation; b) the formation of “ate” complex; c) initiation and propagation; d) termination and e) side reactions.

FIG. 12: FIG. 12 shows SEC traces of EG-GME copolymers synthesized in toluene at different temperatures.

FIG. 13: FIG. 13 shows MALDI TOF MS of BnO—P(EG-co-GME) synthesized in toluene at 25° C.

FIG. 14: FIG. 14 shows stacked NMR spectra of copolymerization of EO and GME in toluene-d₈ at different times of the copolymerization.

FIG. 15: FIG. 15 shows composition profile based on obtained reactivity ratios.

FIG. 16: FIG. 16 shows turbidity measurements of P(EG-co-GME) with different mol % GME at a constant concentration.

FIG. 17: FIG. 17 shows the synthesis of P(EG-co-GME) with different end groups.

FIG. 18: FIG. 18 shows SEC traces of EO-GME copolymers with different end groups.

FIG. 19: FIG. 19 shows SEC traces of α,ω-OH—P(EG-co-GME) and two PLLA-b-P(EG-co-GME)-b-PLLA.

FIG. 20: FIG. 20 shows ¹H NMR spectrum of PLLA-b-P(EG-co-GME)-b-PLLA.

FIG. 21: FIG. 21 shows ¹H NMR DOSY spectrum of PLLA-b-P(EG-co-GME)-b-PLLA.

### EXAMPLES

### Reagents

Chemicals were purchased from TCI, Acros Organics, Roth, Sigma Aldrich and Honeywell, unless otherwise noted. Ethylene oxide was obtained from Air Liquide. Deuterated solvents were purchased from Deutero GmbH. THF was flashed over basic aluminum oxide before usage. Glycidyl methyl ether was dried over CaH₂ and cryo-transferred before the polymerizations.

### Measurements

¹H and ¹³C NMR spectra were recorded on a Bruker Avance III HD 300 spectrometer with 300 MHz and referenced internally to residual proton signals of the deuterated solvent. In case of ¹H DOSY NMR, spectra were recorded on a Bruker Avance III HD 400 spectrometer with 400 MHz and referenced internally to residual proton signals of the deuterated solvent.

M_{w}, Mₙ and dispersities (M_{w}/Mₙ=PDI) of all samples were determined from the corresponding size exclusion chromatograms (refractive index (RI) detector, DMF, calibration with PEG standards).

Size-exclusion chromatography (SEC) were performed with dimethylformamide (DMF with 1 g/L LiBr) as the mobile phase (flow rate 1 mL/min) on poly(2-hydroxyethylmethacrylat) (PHEMA) 300/100/40 columns at 50° C. Polymer concentrations were 1 mg/mL. Calibration was carried out using poly(ethylene glycol) standards (from Polymer Standard Service, Mainz, Germany).

Differential scanning calorimetry (DSC) measurements were carried out in the temperature range of −100 to 100° C. with a heating rate of 10 K/min at a PerkinElmer DSC 8500. The thermal history of the samples was excluded via two cooling and two heating cycles. For each sample, the glass transition and the melting temperatures were obtained from the second heating curve.

MALDI-ToF MS measurements were carried out at a Bruker autoflex max MALDI-TOF/TOF. The potassium salt of trifluoroacetic acid and trans-2-[3-(4-tert-Butylphenyl)-2-methyl-2-propenylidene]malononitrile (DCTB) were utilized as ionization salt and matrix, respectively.

### Example 1: Synthesis of Glycidyl methyl Ether (GME)

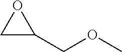 [0114] a) Allyl methyl ether (10.0 g, 139 mmol) was dissolved in 274 mL dichloromethane (DCM) and m-chloroperoxybenzoic acid (m-CPBA, 70%, 37.6 g, 153 mmol (based on m-CPBA)) was added to the solution. After stirring overnight, the solution was filtrated and slowly concentrated under reduced pressure. Crude glycidyl methyl ether was separated from solid impurities via cryo-transfer. Slow evaporation of residual dichloromethane in vacuo gave pure glycidyl methyl ether as a colorless liquid; Yield 41%.
b) 1-Chloro-3-methoxy-propan-2-ol (3.00 g, 2.56 mL, 24.1 mmol) and anhydrous sodium sulfate (1.02 g, 7.23 mmol) were added to a flask equipped with a magnetic stirrer and cooled with a water bath. Finely grounded sodium hydroxide (1.25 g, 31.3 mmol) was added under stirring. After complete reaction (TLC control) the crude product was cryo-transferred in vacuo from the reaction flask and dried over CaH2 under cooling. After an additional cryo-transfer, GME was obtained as a colorless liquid with a yield of 93%.

### Example 2: General Procedure for the Preparation of Random Copolymers Synthesis of α-BzO-P(EG_{0.51}-co-GME_{0.49})

Ph-CH₂—O—CH₂—CH₂—O—[CH₂—CH₂—O—]_{0.51} [symbol] [CH₂CH(CH₂—O—CH₃)—O—]_{0.49}H

Cesium hydroxide monohydrate (28.0 mg, 118 μmol) was dissolved in a THF-water mixture and transferred into a reaction flask, equipped with a Teflon stopcock and a septum. 2-Benzyloxy-ethanol (20.0 mg, 18.7 μL, 131 μmol) was dissolved in benzene and added to the reaction flask. After slow evaporation of the solvent, the resulting solid was dried at 60° C. under high vacuum overnight. The residue was dissolved in DMSO (5 mL) and the solution was cooled to −78° C. GME (367 mg, 374 μL, 4.16 mmol) was added via syringe and EO (183 mg, 189 μL, 4.16 mmol) was cryo-transferred from a graduated ampule into the reaction flask. The solution was stirred for 48 h at room temperature under vacuum. Afterwards, the solution was poured into excess chloroform and the organic phase was extracted against water (3 times) and brine, dried over MgSO₄ and filtrated. P(EG_{0.51}-co-GME_{0.49}) was obtained in quantitative yield as a viscous liquid after evaporation of the solvent and drying under high vacuo. The data of the product can be found in Tables 2 and 3, entry e.

### Example 3: Synthesis of mPEG-b-PGME

Me—O—[CH₂—CH₂—O—]_{0.78}—[CH₂CH(CH₂—O—CH₃)—O—]_{0.22}H

Potassium tert-butoxide (13.0 mg, 118 μmol) was dissolved in a tetrahydrofuran (THF)—water mixture and transferred into a reaction flask, equipped with a Teflon stopcock and a septum. Methoxypoly(ethylene glycol) (mPEG, Mₙ 2 kg/mol; 250 mg, 125 μmol) was dissolved in benzene and added to the reaction flask. After slow evaporation, the residue was dried at 80° C. under high vacuum overnight. The residue was dissolved in dimethyl sulfoxide (DMSO, 5 mL) and the solution was cooled to −78° C. Glycidyl methyl ether (253 mg, 258 μL, 2.88 mmol) was cryo-transferred into the reaction flask. The solution was heated to 55° C. and stirred for 24 h under vacuum. Afterwards, the solution was poured into excess chloroform and the organic phase was extracted against water (3 times) and brine, dried over MgSO₄ and filtrated. mPEG-b-PGME 4 k was obtained as a colorless solid after precipitation in ice-cold diethyl ether; yield quantitative. Table 2 and 3, entry I shows the data for this polymer.

### Example 4: Synthesis of α-BzO-ω-N-succinimidyl carbonate-P(EG-co-GME)

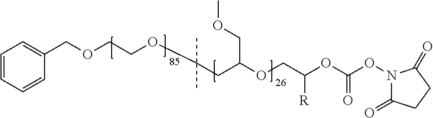

R═H, CH₂OCH₃ N,N′-disuccinimidyl carbonate (3.5 mg, 13.8 μmol) is added to a stirred solution of α-BzO-P(EG-co-GME) (50.0 mg, 4.6 μmol) from Example 2 in dry CH₃CN (1 ml) at room temperature for 18 h. The reaction mixture was dissolved in dichloromethane extracted with a saturated NaHCO₃, water and brine, dried over MgSO₄ and filtered. The solvent was removed under reduced pressure and the polymer dried under vacuum. The product was obtained as a white solid; yield quantitative.

### Example 5: Conjugation of α-BzO-ω-N-succinimidyl carbonate-P(EG-co-GME) to Bovine Serum Albumin (BSA)

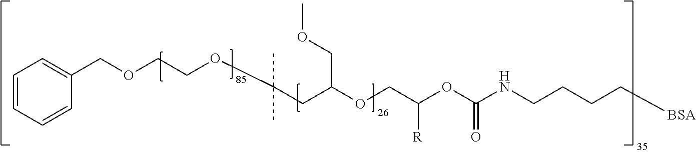

R═H, CH₂OCH₃ Bovine serum albumin (5 mg, 0.07 μmol) and α-BzO-ω-N-succinimidyl carbonate-P(EG-co-GME) (16.9 mg, 2.6 μmol) from Example 4 were stirred in phosphate-buffered saline (PBS buffer) for 1 h. The unreacted polymer was removed by dialysis in deionized water with a 25 kDa membrane filter. The conjugate was then dried by lyophilization and obtained in quantitative yield. In the preparation of this conjugate, the outer lysing group (30-35) are targeted for the conjugation.

### Example 6: Competitive Enzyme-Linked Immunosorbent Assay (ELISA)

The interaction of the copolymer samples entry c, d and e were evaluated by a competitive PEG ELISA kit using a murine monoclonal, horseradish peroxidase conjugated anti PEG antibody (HRP anti-PEG) (Life diagnostics, West Chester, PA, USA). Samples of concentrations ranging from 0 to 4600 μg ml-1 were prepared in dilution buffer. Additionally, mPEG with a molecular weight of 5000 g mol-1 was utilized as internal standard for comparison. 50 μl of each prepared sample was dispensed to a PEG pre-coated 96-well plate and 50 μl of HRP anti-PEG was added to each well. The solutions were incubated for 1 h at 25° C. with a micro-plate shaker and then washed six times with 400 μl of wash buffer per each well. After removal of residual droplets, 100 μl of 3,3′,5,5′-Tetramethylbiphenyl-4,4′-diamine was added to each well and the solutions were mixed on a micro-plate shaker for 20 min. The reaction was stopped by addition of 100 μl of stop solution and the absorbance at 450 nm was read within 5 minutes.

Analysis of ELISA Data: The determined absorbance values were normalized to visualize the percent of maximal binding. The sample concentrations were transformed to a function of log₁₀. The sigmoidal fits were calculated using the following equation with a representing the upper, b the lower limit, c the inflection point and d the hill slope.

$$y = a + ( b - a ) / 1 + 1 {0}^{( c - x ) * d}$$

### Example 7: Kinetic of the Synthesis of α-BzO-P(EG-co-GME)

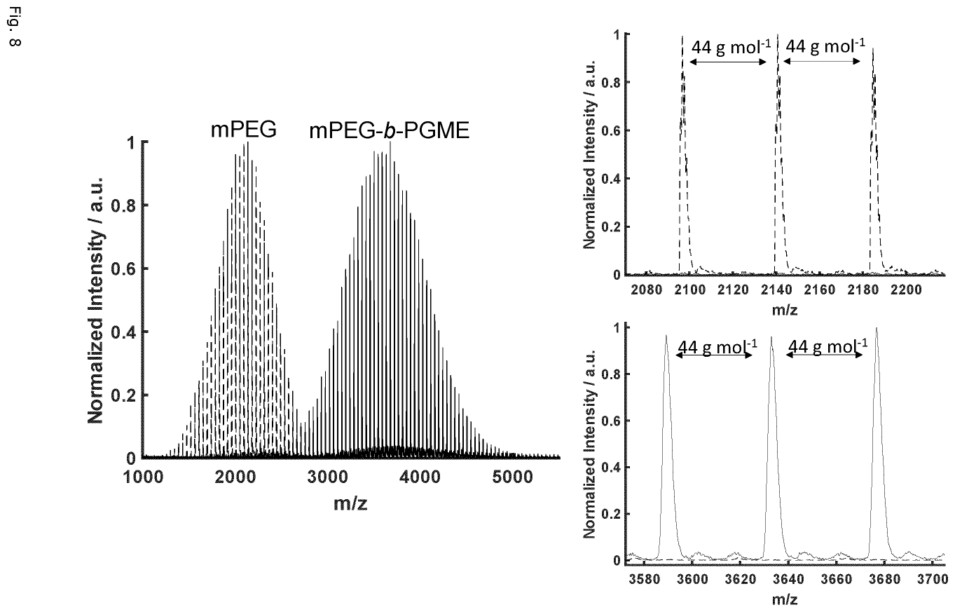

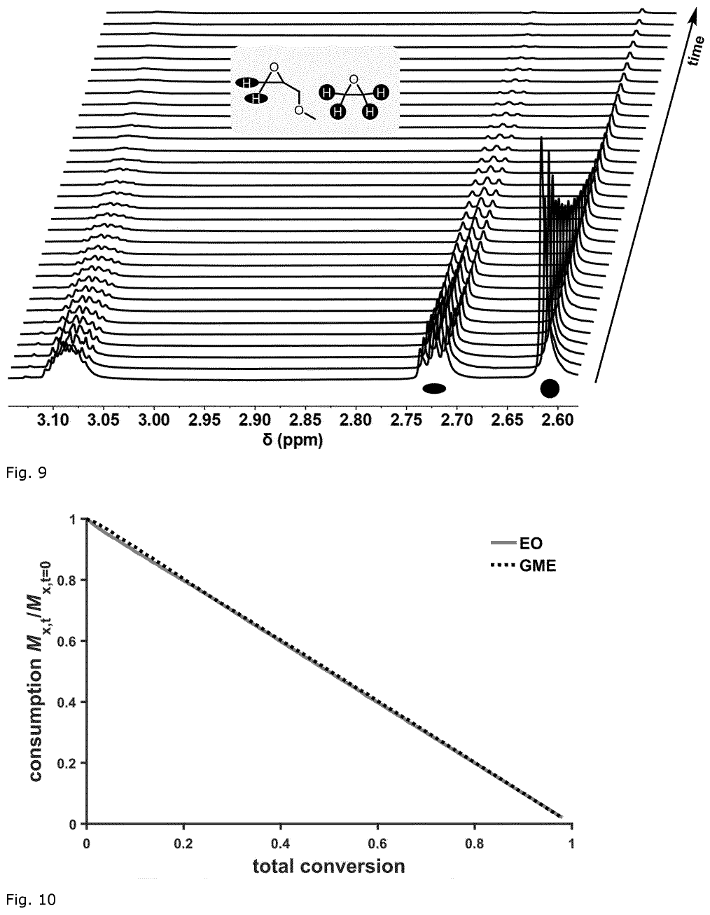

The synthesis of α-BzO-P(EG-co-GME) was repeated in fully deuterated DMSO for an online in situ ¹H NMR kinetic measurement. 95 mg glycidyl methyl ether (95 μl, 1.1 μmol) and 32 mg ethylene oxide (33 μl, 7.1 μmol), i.e. 60% glycidyl methyl ether and 40% ethylene oxide where solved in fully deuterated DMSO. The consumption of ethylene oxide and glycidyl methyl ether over time was measured by ¹H-NMR spectroscopy. As measure for the ethylene oxide concentration, the signal of the protons of ethylene oxide at about 2.6 ppm was used. For glycidyl methyl ether the signals of the protons at the oxirane ring with absorptions at about 2.75 and 3.1 ppm were used. FIG. 9 shows the decrease of the selected ¹H NMR signals of the two monomers over time. FIG. 10 shows plot of monomer consumption M_{x,t}/M_{x,t=0} versus total conversion. As can be seen, ethylene oxide and glycidyl methyl ether are consumed at exactly the same rate. This data shows that the copolymers of the present invention are almost ideal random copolymers.

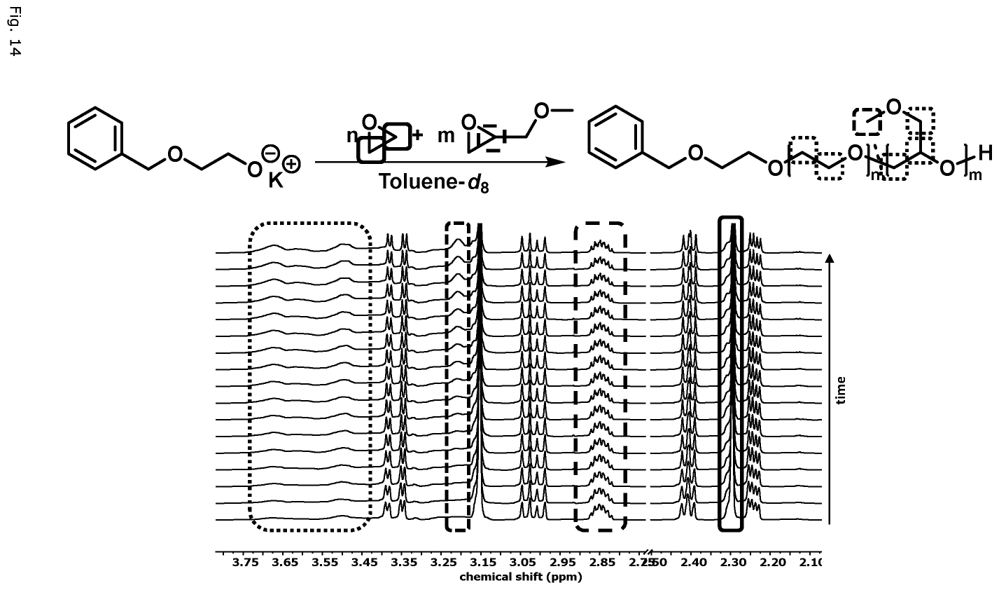

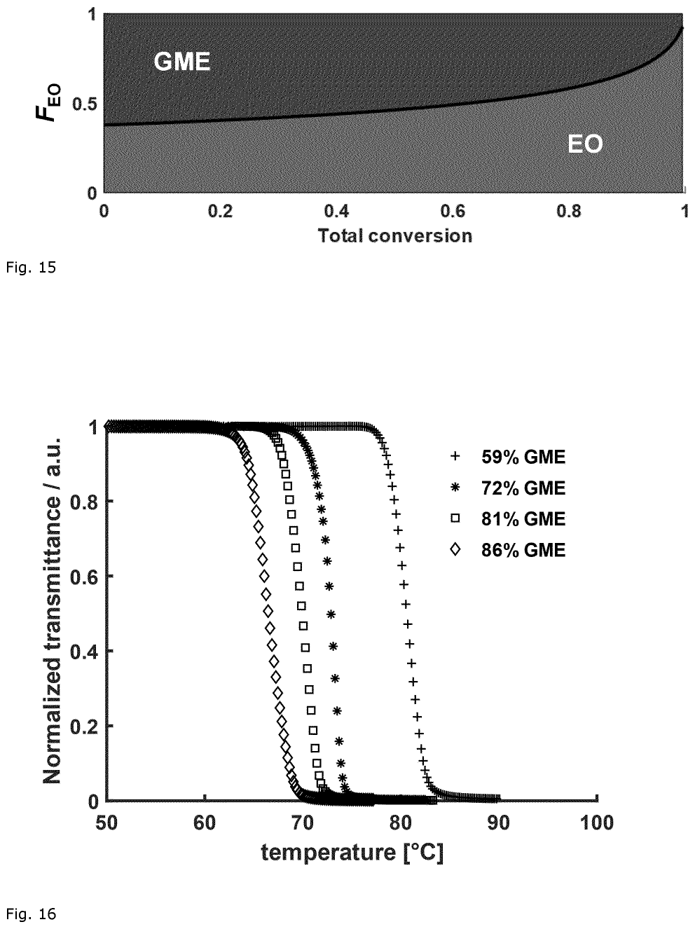

Further, in-situ ¹H NMR kinetics were conducted to investigate the influence of the solvent change from DMSO to toluene on the copolymerization kinetics as can be seen in FIG. 14. In addition, based on the decrease of the monomer signals during copolymerization, copolymerization parameters of r_{EO}=0.61 and r_{GME}=1.65 were obtained, showing a slighty preferred incorporation of GME repeating units over EO at the beginning of the copolymerization due to the higher reactivity of GME over EO leading to a statistical copolymer as can be seen in FIG. 15.

### Example 8: Synthesis of mP(EG_{0.66}-co-GME_{0.34}) (1) according to FIG. 17

mP(EG_{0.66}-co-GME_{0.34}) (1): Diethylene glycol monomethyl ether (50.8 mg, 420 μmol) was dissolved in benzene (5 mL) and transferred via syringe into a flame-dried flask under stationary vacuum. Potassium-tert-butoxide (42.7 mg, 380 μmol) was dissolved in stabilizer-free THF (3 mL) and small quantities of Millipore water and transferred into the flask. High vacuum was applied to the flask and the solvents were removed under high vacuum. The resulting initiator salt was dried under high vacuum at 60° C. overnight and finally dissolved in dry DMSO (5 mL) under stationary vacuum. After freezing the resulting solution at −90° C., GME (1.03 g, 11.6 mmol) were added via syringe to the flask. EO (1.04 g, 1.00 mL, 23.6 mmol) was added to the flask via cryo-transfer before the cooling bath was removed and the reaction mixture was allowed to warm up to room temperature. The reaction was allowed to stir for 72 h at room temperature. Subsequently, a mixture of 2M HCl (500 μL) and MeOH (500 μL) was added to terminate the polymerization.

The reaction mixture was added to chloroform (20 mL), washed three times with Millipore water (10 mL) and once with brine. The organic phase was dried over MgSO4 before the solvent was removed under vacuum. The resulting polymer was dialyzed against MeOH (MWCO of 2 kDa) for 24 h and freeze-dried under high vacuum over night to give 1.82 g (86%) of the polymer (1) of FIG. 17 as a viscous pale-yellow liquid. The data of the product can be found in Tables 2 and 3, entry n.

Further, α-BzO-P(EG-co-GME) with varying amounts of the monomers were prepared according to the same procedure with a polymerization temperature of 55° C. and benzyloxy ethanol as initiator, respectively, instead of diethylene glycol monomethyl ether (entries c and d of Tables 2 and 3).

### Example 9: Synthesis of mP(EG_{0.66}-co-GME_{0.34})-Ms (2) according to FIG. 17

mP(EG_{0.66}-co-GME_{0.34})-Ms (2): Polymer (1) (600 mg, 120 μmol) was dissolved in benzene (5 mL) and added to a flame-dried flask under argon-flow. Benzene was slowly removed under high vacuum. The polymer was heated to 60° C. and dried under high vacuum overnight. Under argon-flow, the dry polymer was dissolved in dry DCM (10 mL) and triethyl amine (97.1 mg, 960 μmol) and methanesulfonyl chloride (110 mg, 960 μmol) were added under ice-cooling. The mixture was allowed to stir for 72 h at room temperature under argon. Afterwards, the mixture was washed three times with Millipore water and once with brine. The organic phase was dried with MgSO4 and the solvent was removed under high vacuum to give 427 mg (70%) of the mesylated polymer (2) as a viscous dark yellow liquid. The data of the product can be found in Tables 2 and 3, entry o.

### Example 10: Synthesis of mP(EG_{0.66}-co-GME_{0.34})-N₃ (3) according to FIG. 17

Polymer (2) (400 mg, 80.0 μg) was dissolved in benzene (5 mL) and added to a flame-dried flask under argon-flow. Benzene was removed under high vacuum. The polymer was further heated to 60° C. and dried under high vacuum overnight. Subsequently, the dry polymer was dissolved in dry DMF (8 mL) and sodium azide (41.6 mg, 640 μmol) was added as a suspension in dry DMF (2 mL) under argon-flow. The reaction mixture was stirred at 65° C. for 72 h. The solvent was removed under high vacuum and the residue was suspended in DCM (10 mL) and treated in an ultrasonic bath for 20 min. The remaining residue was filtered off and the filtrate was washed three times with Millipore water and once with brine. The organic phase was dried over MgSO₄ and the solvent was removed to give 284 mg (71%) of polymer (3) as a viscous dark yellow liquid. The data of the product can be found in Tables 2 and 3, entry p.

### Example 11: mP(EG_{0.66}-co-GME_{0.34})-NH₂ (4) according to FIG. 17

Polymer (3) (180 mg, 40.0 μg) was dissolved in EtOH (10 mL). Catalytic amounts of Pd/C 10 wt % (13.8 mg, 10.0 μmol of Pd) were added to the solution and a balloon filled with an excess of H2-gas was connected to the flask. Vacuum was applied to the flask until the solvent started to evaporate and the flask was subsequently flushed with argon. This process was repeated two more times. Afterwards, vacuum was applied until the solvent started to evaporate and the flask was flushed with H2-gas. The last step was repeated two more times. The reaction was allowed to stir under 1 atm of H2-gas for 48 h. After 24 h the H2 balloon was refilled to guarantee a saturation of the atmosphere with H2. After 48 h the balloon was removed, the catalyst was filtered off over zeolite and the solvent was removed under high vacuum to give 142 mg (79%) of the amino-terminated polymer (4). The data of the product can be found in Tables 2 and 3, entry q.

### Example 12: Synthesis of α,ω-OH—P(EG-co-GME)

2,2′-((Propane-2,2-diylbis(4,1-phenylene))bis(oxy))bis(ethan-1-ol) (BHEPP) (123 mg, 389 μmol) was dissolved in benzene (5 mL) and transferred via syringe into a flame-dried flask under argon. Potassium-tert-butoxide (43.6 mg, 389 μmol) was dissolved in stabilizer-free THF (3 mL) and small quantities of Millipore water and transferred into the flask. High vacuum was applied to the flask and the solvents were removed under high vacuum. The resulting initiator salt was dried under high vacuum at 70° C. overnight and finally dissolved in dry DMSO (5 mL) under stationary vacuum. After freezing the resulting solution at −78° C., GME (873 mg, 890 μL, 9.91 mmol) were added via syringe to the flask. EO (1.46 g, 1.50 mL, 33.1 mmol) was added to the flask via cryo-transfer before the cooling bath was removed and the reaction mixture was allowed to warm up to room temperature. The reaction was allowed to stir for 22 h at 55° C. Dowex® (30 mg) and water (4 mL) were added to the solution after venting the flask. After dialysis against water and lyophilization, the polymer was obtained as a viscous liquid (1.36 g, 57%). The data of the product can be found in Tables 2 and 3, entry r.

### Example 13: Synthesis of PLLA₆₉-b-P(EG-co-GME)-b-PLLA₇₀

In a glove box, dry α,ω-OH—P(EG-co-GME) (400 mg, 66.7 μmol) and L-Lactide (577 mg, 4.00 mmol) were dissolved in dry DCM (3 mL). DBU (6.08 mg, 40.0 μmol) dissolved in DCM (70.0 μL) was added and the resulting solution was stirred for 30 min. After addition of benzoic acid (24.4 mg, 200 μmol) in DCM (3 mL), the crude polymer was precipitated in cold diethyl ether. The last step was repeated two times. The block copolymer was obtained after lyophilization from benzene as a colorless solid (79%).

### Example 14: Synthesis of α-BzO-P(EG_{0.50}-co-GME_{0.50}) in toluene

Other copolymerization conditions, but temperature, and solvent were held constant compared to Example 8 to merely investigate the influence of temperature and solvent on the copolymerization. In the investigated temperature range (25-50° C.), the GME-EO copolymerization in toluene results in well-defined copolymers with low dispersities <1.10 and monomodal narrow molecular weight distributions as can be seen in FIG. 12.

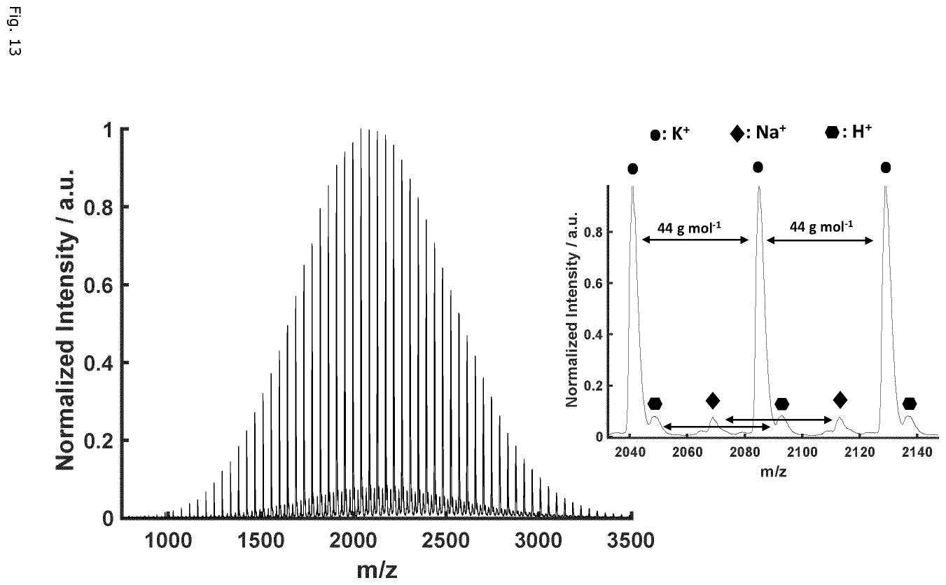

The MALDI-TOF MS of α-BzO-P(EG_{0.50}-co-GME_{0.50}) (toluene, 25° C.) shows a clear overlay of theoretical and experimental Mₙ values (2 kg mol⁻¹) and reveals an exceptional high end-group fidelity (>99%), known from the copolymerization in DMSO as can be seen in FIG. 13.

The data of the product can be found in Tables 2 and 3, entry s to u.

### Discussion of the Results

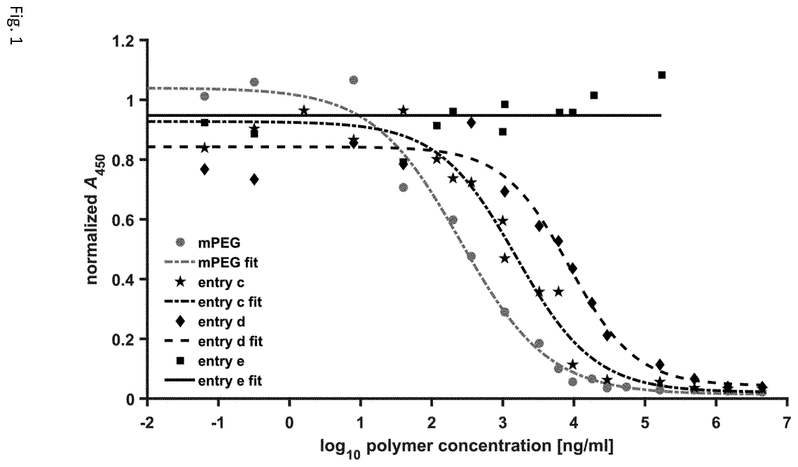

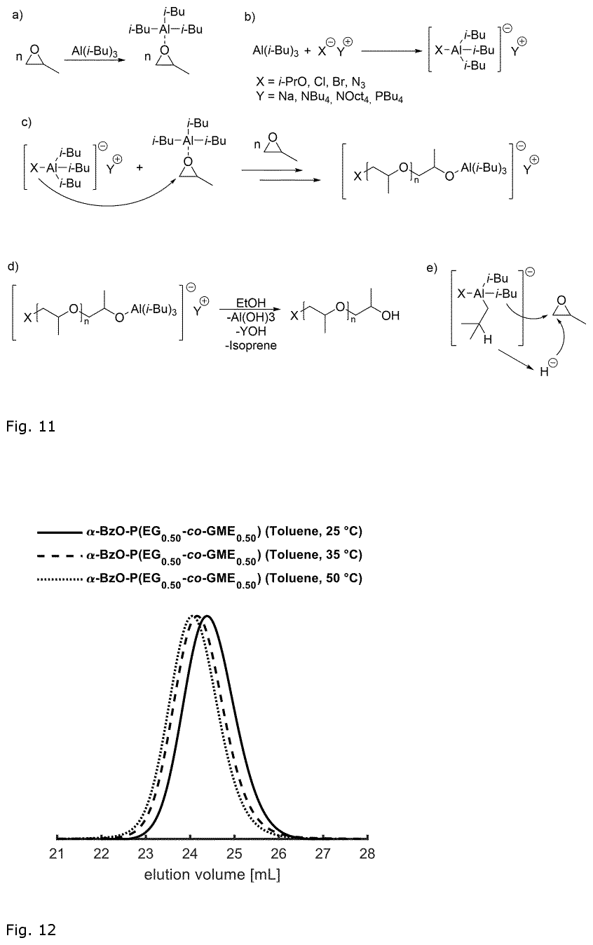

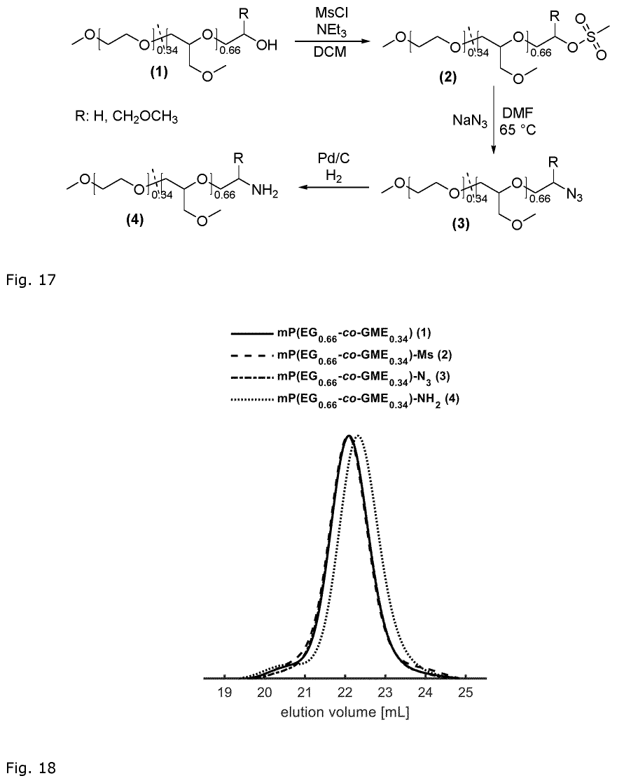

Tables 2, 3, 4 and 5 and FIGS. 1 to 21 show the properties of the polymers prepared. They are discussed in the following.

TABLE 2
Composition of polymers prepared

| Entry | | | | | |
|---|---|---|---|---|---|
| (Example) | Sample | DP_{EO NMR} | DP_{GE NMR} | mol %_{EO NMR} | mol %_{GME NMR} |
| c | α-BzO P(EG_{0.84}-co-GME_{0.16}) | 112 | 21 | 84 | 16 |
| (9) | | | | | |
| d | α-BzO P(EG_{0.76}-co-GME_{0.24}) | 85 | 26 | 76 | 24 |
| (9) | | | | | |
| e | α-BzO P(EG_{0.51}-co-GME_{0.49}) | 35 | 33 | 51 | 49 |
| (9) | | | | | |
| l | α-MeO—PEG_{0.88}-b-PGME_{0.22} | 50 | 14 | 78 | 22 |
| (6) | | | | | |
| n | α-MeO—P(EG_{0.66}-co-GME_{0.34}) | 56 | 29 | 66 | 34 |
| (8) | | | | | |
| o | α-MeO-ω-Ms—P(EG_{0.66}-co-GME_{0.34}) | 56 | 29 | 66 | 34 |
| (9) | | | | | |
| p | α-MeO-ω-N₃—P(EG_{0.66}-co-GME_{0.34}) | 56 | 29 | 66 | 34 |
| (10) | | | | | |
| q | α-MeO-ω-NH₂ P(EG_{0.66}-co-GME_{0.34}) | 56 | 29 | 66 | 34 |
| (11) | | | | | |
| r | α,ω-OH—P(EG-co-GME) | 92 | 28 | 77 | 23 |
| (12) | | | | | |
| s | α-BzO—P(EG_{0.50}-co-GME_{0.50}) | 15 | 15 | 50 | 50 |
| (14) | (toluene, 25° C.) | | | | |
| t | α-BzO—P(EG_{0.50}-co-GME_{0.50}) | 15 | 15 | 50 | 50 |
| (14) | (toluene, 35° C.) | | | | |
| u | α-BzO—P(EG_{0.50}-co-GME_{0.50}) | 15 | 15 | 50 | 50 |
| (14) | (toluene, 50° C.) | | | | |

GE = Glycidyl ether;
DP = degree of polymerization

TABLE 3
Characterization data of polymers prepared

| Entry/ | | M_{n, NMR} | M_{n, MALDI} | M_{n, SEC} | | End-group |
|---|---|---|---|---|---|---|
| (Example) | Sample | [kg/mol] | [kg/mol] | [kg/mol] | PDI_{SEC} | fidelity_{MALDI} |
| c (9) | α-BzO P(EG_{0.84}-co-GME_{0.16}) | 6.9 | 4.9 | 3.7 | 1.05 | >99% |
| d (9) | α-BzO P(EG_{0.76}-co-GME_{0.24}) | 6.0 | 5.2 | 3.8 | 1.06 | >99% |
| e (9) | α-BzO P(EG_{0.51}-co-GME_{0.49}) | 4.2 | 3.8 | 2.4 | 1.09 | >99% |
| l (6) | α-MeO—PEG_{0.88}-b-PGME_{0.22} | 3.6 | 3.7 | 2.8 | 1.05 | >99% |
| n (8) | α-MeO—P(EG_{0.66}-co-GME_{0.34}) | 5.0 | 5.0 | 3.9 | 1.08 | >99% |
| o (9) | α-MeO-ω-Ms—P(EG_{0.66}-co-GME_{0.34}) | 5.0 | 5.1 | 3.9 | 1.09 | >99% |
| p (10) | α-MeO-ω-N₃—P(EG_{0.66}-co-GME_{0.34}) | 5.0 | N/A | 3.9 | 1.07 | N/A |
| q (11) | α-MeO-ω-NH₂ P(EG_{0.66}-co-GME_{0.34}) | 5.0 | N/A | 4.0 | 1.09 | N/A |
| r (12) | α,ω-OH—P(EG-co-GME) | 6.0 | 6.1 | 4.3 | 1.09 | >99% |
| s (14) | α-BzO—P(EG_{0.50}-co-GME_{0.50}) | 2.1 | 2.1 | 1.6 | 1.06 | >99% |
| | (toluene, 25° C.) | | | | | |
| t (14) | α-BzO—P(EG_{0.50}-co-GME_{0.50}) | 2.2 | 2.2 | 1.8 | 1.07 | >99% |
| | (toluene, 35° C.) | | | | | |
| u (14) | α-BzO—P(EG_{0.50}-co-GME_{0.50}) | 2.2 | 2.2 | 1.9 | 1.07 | >99% |
| | (toluene, 50° C.) | | | | | |

Mₙ = molecular weight (number average molar mass), PDI = dispersity.

TABLE 4
Thermal properties

| | | | | Cloud |
|---|---|---|---|---|
| | [GME] | Mₙ^{b} | | Point |
| Sample | (mol % )^{a} | (kg/mol) | PDI^{c} | (° C.) |
| α-BzO—P(EG_{0.41}-co-GME_{0.59}) | 59 | 4.8 | 1.08 | 81 |
| α-BzO—P(EG_{0.28}-co-GME_{0.72}) | 72 | 4.7 | 1.06 | 73 |
| α-BzO—P(EG_{0.19}-co-GME_{0.81}) | 81 | 4.8 | 1.10 | 70 |
| α-BzO—P(EG_{0.14}-co-GME_{0.86}) | 86 | 4.8 | 1.07 | 66 |

^{a}calculated via ¹H NMR spectroscopy
^{b}determined by MALDI-ToF mass spectrometry
^{c}determined by SEC (DMF, calibration with PEG standards)

Table 4 shows the thermal properties of the respective compounds. The polymers were prepared according to Example 8 with varying amounts of the monomers and with benzyloxy ethanol as initiator, respectively, instead of diethylene glycol monomethyl ether. The cloud point of the random copolymers of EO and GME was investigated via turbidimetry measurements of copolymers containing high amounts (≥ 59 mol %) of GME. Cloud point was defined at a transmittance of 50%. Benzyloxy-initiated copolymers synthesized via anionic ring-opening polymerization (AROP) in DMSO were used for the measurements.

The cloud point of the copolymers decreases with increasing amount of GME in the polymer backbone. Despite the decrease in the cloud point with increasing mol % GME, solubility in water under physiologically conditions is ensured for all copolymers.

### Immunogenicity

As can be seen from Table 3, entries c to e and I show copolymers of the present invention which consisting of ethylene oxide repeating units and glycidyl methyl ether repeating units with about the same molecular weight, i.e. with an Mₙ of about 4 to 5 kg/mol. The amount of glycidyl methyl ether repeating units is increasing from c to e from 16% to 49% (see Table 2, entries c to e and l). FIG. 1 shows the ELISA test results of polymers c to e and of commercially available mPEG. It shows the function of the normalized absorption at a wavelength of 450 nm versus the log10 function of the polymer concentration in nanograms per ml, therefore illustrating the anti-PEG antibody interaction with the investigated polymer concentrations. The ELISA data shows that a strong influence of an increasing concentration of alkyl side chains can be observed (it is important to note that the x-axis has a logarithmic scale). With increasing amount of GME incorporated into the polyether structure, significantly higher polymer concentrations are necessary to observe interactions between the copolymer and the APA. Remarkably, at 49 mol % of GME, absolutely no interaction between APA and the copolymer is present. This means that the respective copolymer cannot be detected by APAs, i.e. it is not immunogenic.

### Dispersity

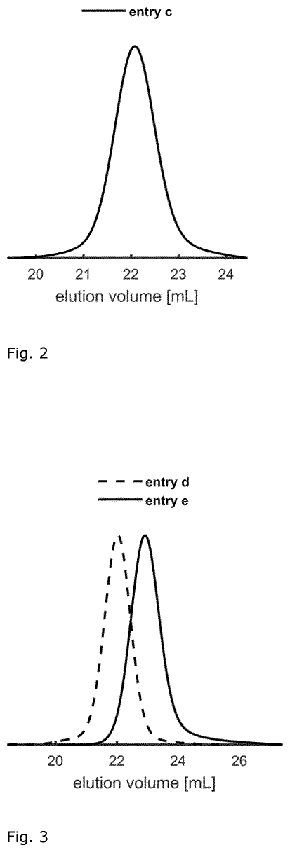

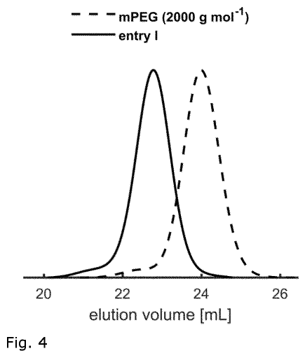

As can be seen from Table 3, entries c to e and l, all polymers of the present invention have a very low dispersity, i.e. 1.10 or lower. For visualization, the SEC traces of these polymers are shown in FIGS. 2 to 4 and 12. FIG. 4 shows in addition the SEC trace of mPEG. It can clearly be seen that the present invention provides polymers with a very narrow molecular weight distribution.

### Preparation and Purity of the Polymers of the Present Invention

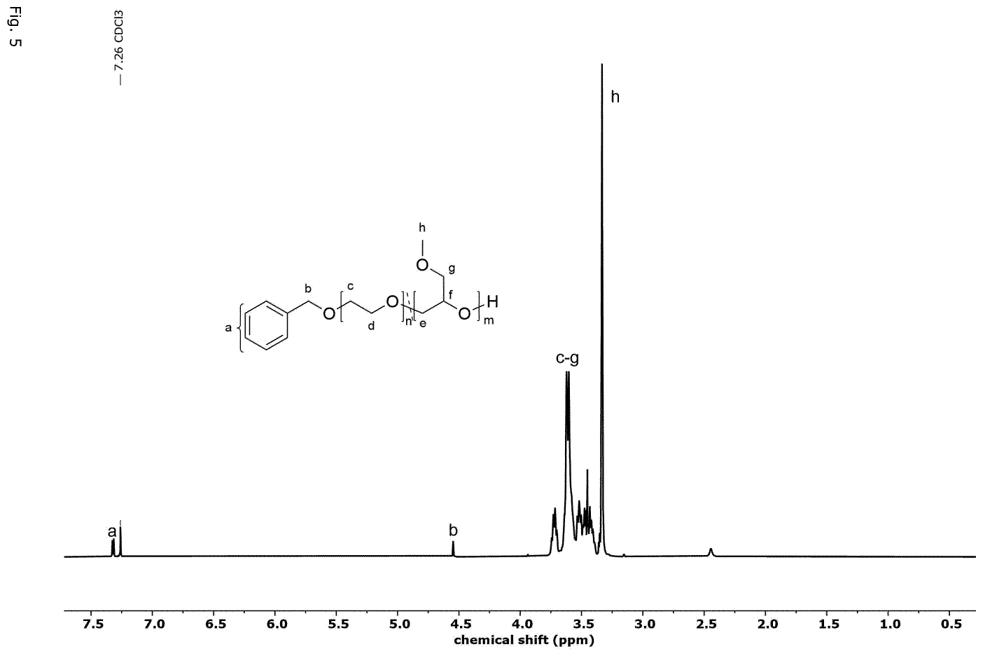

FIG. 5 shows the ¹H NMR spectrum of α-BzO P(EG_{0.51}-co-GME_{0.49}), i.e., the polymer of Example 2 (entry e of Tables 2 and 3). The product is as obtained from Example 2, without further purification. As can be seen, the present method does not only provide polymers of narrow molecular weight distribution, but also very pure polymers without the need for laborious purification steps. This is an important feature for the use in pharmaceutical applications.

The synthesis of bifunctional initiated α,ω-OH—P(EG-co-GME) copolymers enables the synthesis of ABA triblock copolymers. In this case, P(EG-co-GME) bearing two hydroxyl-termini (Example 12) was used as an macroinitiator for ring-opening polymerization of L-lactide, enabling the synthesis of PLLA-b-P(EG-co-GME)-b-PLLA, wherein PLLA is

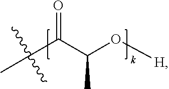

The polymerization is catalyzed by the organic base DBU (1,8-Diazabicyclo [5.4.0]undec-7-ene).

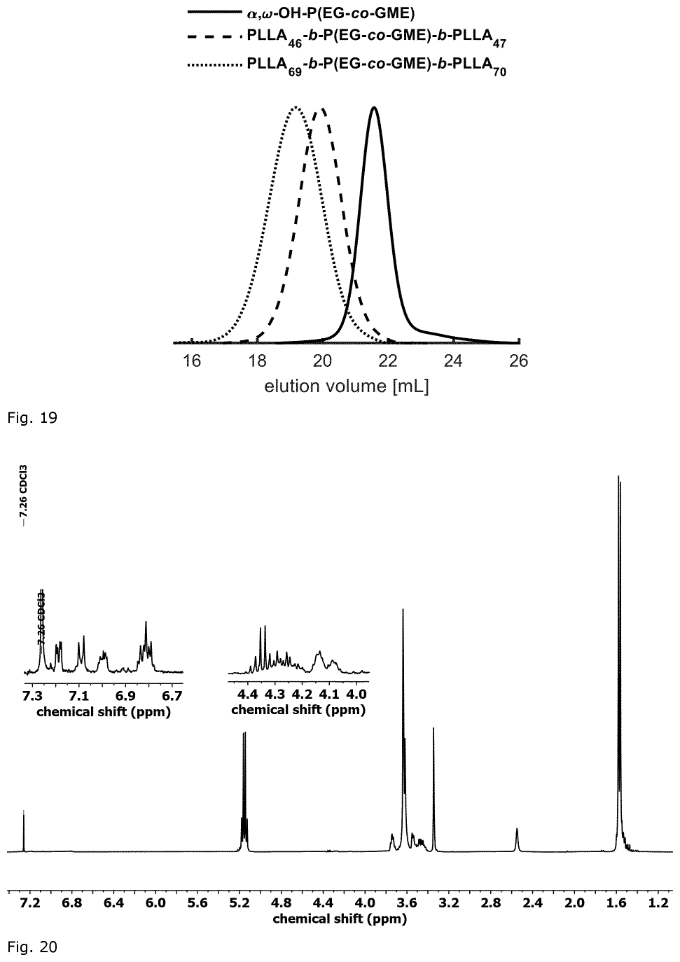

FIG. 19 shows an overlay of the SEC traces of the macroinitiator α,ω-OH—P(EG-co-GME) (polymer of Example 12) and two PLLA-b-P(EG-co-GME)-b-PLLA (PLLA₄₆-b-P(EG-co-GME)-b-PLLA₄₇ is polymer of Example 13) with different degrees of polymerization (k=46, 47 and 69, 70), confirming successful chain extension from the macroinitiator.

FIG. 20 shows the 1H NMR sprectrum of Example 13, confirming the successful polymerization of L-lactide.

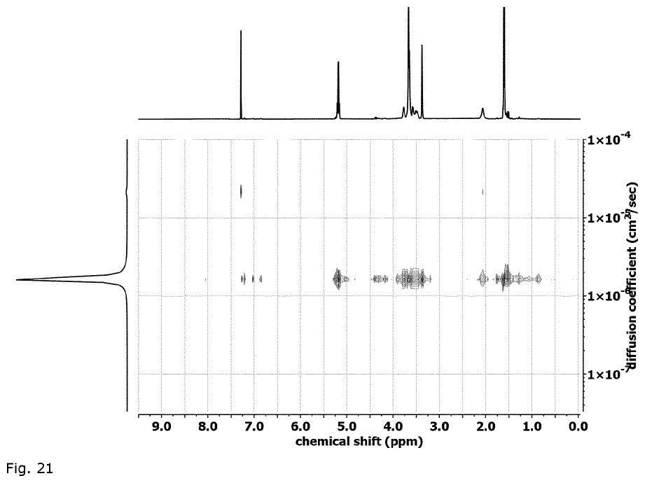

FIG. 21 shows the ¹H DOSY NMR confirming that the blocks within the copolymer are linked via a covalent bond.

### End-Group Fidelity

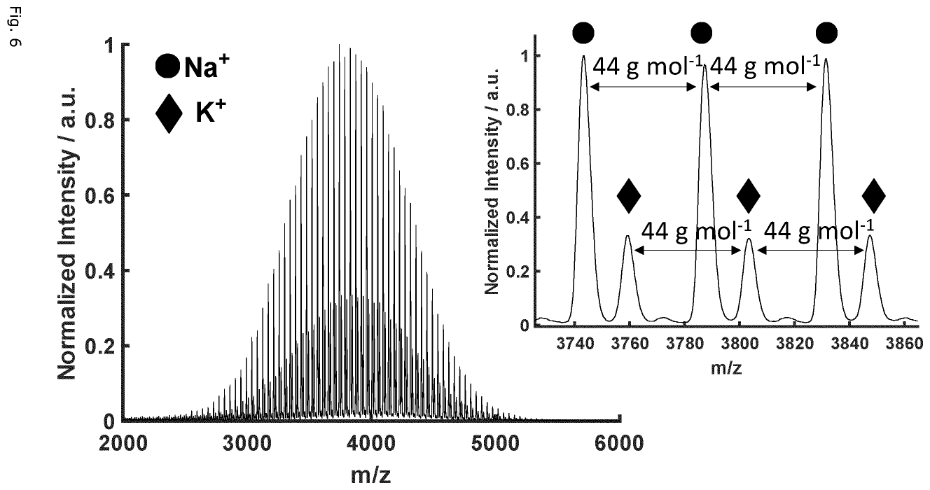

As can be seen from Table 3, the end-group fidelity for all polymers of the present invention is nearly 100%. FIG. 6 shows the MALDI TOF mass spectrum of α-BzO P(EG_{0.51}-co-GME_{0.49}), i.e. the polymer of Example 2 (entry e of Tables 2 and 3) with potassium and sodium cations. The only peaks visible here are the peaks of the product molecule. There are no peaks form macromolecules with a different end group. This shows that end-group fidelity of the polymers of the present invention is very high. In addition, the spectrum confirms the narrow molecular weight distribution.

### Solubility

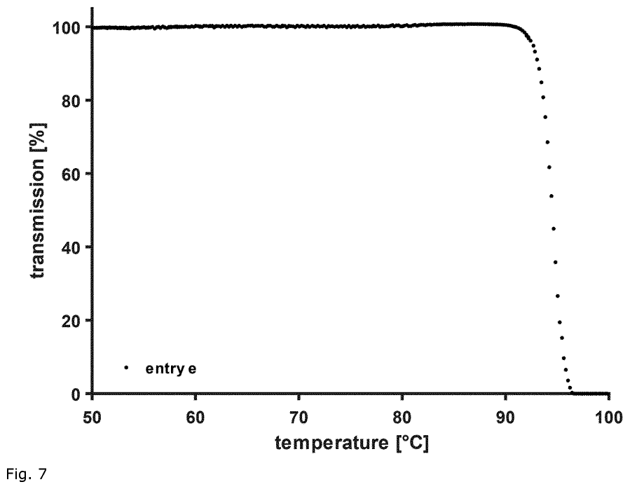

FIG. 7 shows the cloud point measurement of α-BzO-P(EG_{0.51}-co-GME_{0.49}), i.e. the polymer of Example 2 (entry e of Tables 2 and 3). The cloud point determines the temperature at which immiscibility in water/in aqueous solution is observed upon heating. As an example, for the copolymer with 49% GME, the cloud point can be observed at 95° C., showing excellent water solubility of the P(EG-co-GME) copolymers. The homopolymer PEG shows a cloud point of ca. 100° C. All other copolymers of the present invention with 26-49% GME are in between PEG and this copolymer, showing cloud points of 96-100° C. For copolymers of 25 mol % GME incorporation and lower no cloud point was detected in water at temperatures ranging from 0 to 99° C. Thus, GME copolymerization hardly affects water solubility, which is a feature of the similar structure of polyethylene glycol and methyl ethyl ether. This is an important feature of the polymers of the present invention as it shows that they can substitute PEG in aqueous systems without causing problems with phase separation or even precipitation.

### Comparison of Dispersity of End Group Fidelity of the Polymers of the Present Invention With That of mPEG

FIG. 8 shows an overlay of MALDI TOF mass spectra of commercially available mPEG and a block copolymer of the present invention (polymer of Example 3 (entry I of Tables 2 and 3). As can be seen, a clear shift between the macroinitiator mPEG and entry I is observed, indicating a successful block copolymer synthesis. Absence of macroinitiator in the MALDI-ToF MS of entry I further proves quantitative block copolymer formation. This is further proven by a distance of the signals of 44 g mol⁻¹.

### Crystallinity

Table 5 shows thermal properties of some of the polymers of Tables 2 and 3:

| | T_{g} | Tₘ | ΔH_{PEG} | |
|---|---|---|---|---|
| Sample | [° C.] | [° C.] | [J/g] | X_{c, PEG} |
| a | −56 | 46 | 88.63 | 0.45 |
| b | −56 | 45 | 76.79 | 0.39 |
| c | −59 | 37 | 56.26 | 0.29 |
| l | −59 | 45 | 80.16 | 0.41 |
| e | −62 | N/A | N/A | 0.00 |

T_{g} = Glass transition temperature;
Tₘ = Melting point;
ΔH_{PEG} = Enthalpy of fusion;
X_{c} = Crystallinity (all measurements by DSC)I

As can be seen, crystallinity of the inventive polymers is low and can be completely interrupted by the incorporation of GME monomer in the polymer structure. This prevents for example accumulation of the polymers in the kidney or in the liver.

1. Polyether polymers represented by the following formula [I]:
—(CH₂CHR—O—)ₘ— [I]
wherein
10 to 90% of the residues R are hydrogen and 10% to 90% of the residues R are independently from each other selected from the group consisting of, methoxymethyl, ethoxymethyl, n-propoxymethyl and iso-propoxymethyl;
at least 1% of the residues R are methoxymethyl;
up to 50% of the residues R may be selected from the group consisting of ethoxymethyl, n-propoxymethyl and iso-propoxymethyl;
and
m is in the range of from 10 to 1000,
wherein the dispersity is 1.15 or less measured by size exclusion chromatography in DMF by calibration with PEG standards.

2. The polyether polymer according to claim 1, wherein the polyether is a poly(glycidyl methyl ether-co-ethylene oxide) copolymer.

3. The polyether polymer according to claim 1, wherein 30 to 70% of the residues R are hydrogen and the remaining residues R are methoxymethyl.

4. The polyether polymer according to claim 1, wherein the polymer is a random copolymer or a statistical copolymer.

5. The polyether polymers according to claim 1 represented by the following formula [II]:
X—(CH₂CHR—O—)ₘCH₂CHR—Y [II]
wherein X is selected from the group consisting of hydrogen, alkyl, hydroxy, sulfanyl, C1-C10-alkoxy, C1-C10-thioalkoxy, amino, amino-C1-C10-alkoxy, dibenzylamino-C1-C10-alkoxy, amide, N-heterocyclic carbenes and N-heterocyclic olefins;
Y is selected from the group consisting of hydrogen, hydroxy, alkoxy, —CH₂—C(═O)—R wherein R is as defined above, —CHO, alkylcarbonyloxy, alkyoxycarbonyloxy, amine, C1-C10-alkylamine, carboxamido, azide, halogen, sulfanyl, thioalkoxy, N-succinimidyl carbonate and sulfonate;
X and/or Y may also be selected from the group consisting of low molecular weight drugs, nanocarriers, liposomal structures, peptides, polypeptides, proteins, glycoproteins, polynucleotide, polysaccharides, lipid structures, liposomes, surfaces and interfaces, which are bonded directly by a covalent bond or by a spacer to the polymer,
m is in the range of from 9 to 999,
wherein the dispersity is 1.15 or less measured by size exclusion chromatography in DMF by calibration with PEG standards.

6. The polyether polymer according to claim 5, wherein X is alkoxy and Y is hydroxy.

7. The polyether polymer according to claim 5, wherein X and Y are each hydroxy.

8. The polyether polymers according to claim 1 represented by the following formula [II]:
X—(CH₂CHR—O—)ₘCH₂CHR—Y [II]
wherein X is selected from the group consisting of hydrogen or X′,
wherein X′ is selected from the group consisting of 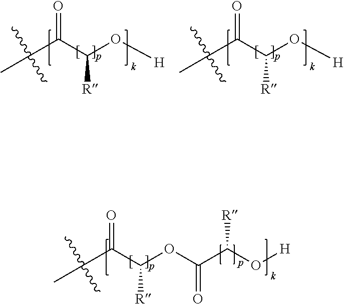
wherein R″═H or alkyl, alkenyl, aryl, alkoxymethyl, alkenyloxymethyl, aryloxymethyl, aminomethyl and thioalkoxymethyl and
p=1, 2, 3, 4 or 5, and
k=5-500; 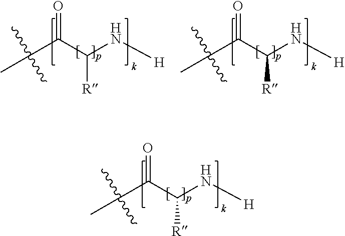
wherein R″═H or alkyl, alkenyl, aryl, alkoxymethyl, alkenyloxymethyl, aryloxymethyl, aminomethyl and thioalkoxymethyl, and
p=1, 2, 3, 4 or 5 and
k=5-500; and 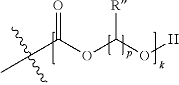
wherein R″═H or alkyl, alkenyl, aryl, alkoxymethyl, alkenyloxymethyl, aryloxymethyl, aminomethyl and thioalkoxymethyl
q=3, 4 or 5 and
k=5-500;
and 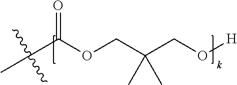
wherein k=5-500; and
wherein Y is Z—W meaning composed of substituent Z and W linked via a covalent bond, wherein
Z is —O—R′—O— and
W is —R—CHCH₂—(O—CHRCH2—)ₘ—V;
wherein V is X or X′;
wherein R′ is selected from the group consisting of 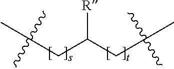
wherein
R″═H or alkyl, alkenyl, aryl, alkoxymethyl, alkenyloxymethyl, aryloxymethyl, aminomethyl and thioalkoxymethyl, and
s=0-20
t=0-20; and
further R′ is selected from 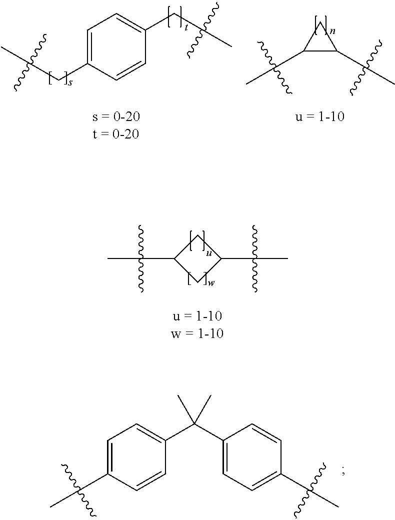
and wherein
m is in the range of from 9 to 999,
wherein the dispersity is 1.15 or less measured by size exclusion chromatography in DMF by calibration with PEG standards.

9. The polyether polymer according to claim 5, wherein end-group fidelity of the polymer in regard to group X and/or in regard to group Y is at least 95%.

10. The conjugate, comprising a polyether polymer of claim 1 and a substrate.

11. The conjugate according to claim 10, wherein the substrate is selected from the groups consisting of low molecular weight drugs, nanocarriers, liposomal structures, peptides, polypeptides, proteins, glycoproteins, polynucleotides, polysaccharides, lipid structures, liposomes, surfaces and interfaces.

12. The process for the preparation of a polyether polymer according to claim 1, by anionic ring-opening copolymerization comprising the steps of:
providing an anion An⁻,
adding at least one monomer of the formula 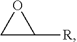
where in R is as defined in claim 1,
allowing the polymerization to proceed at a temperature in the range of −10 to 90° C.
wherein the monomer comprises less than 1 wt % of epichlorohydrin.

13. The process according to claim 12, wherein the step of adding at least one monomer and the step of allowing the polymerization to proceed are repeated at least one time, whereby at least one different monomer is used than the first time.

14. The process according to claim 12, wherein the counter ion to the An⁻ anion is selected from the group consisting of Na⁺, K⁺ and Cs⁺.

15. A process for the preparation of a conjugate of the polymers with a bioactive compound using the polyether polymers according to claim 1.

16. The process according to claim 15 for the preparation of conjugated lipids for use in vaccinesUse according to claim 15 for the preparation of conjugated lipids for use in vaccines.

17. The process according to claim 16 based on lipid nanoparticles.

18. The process according to claim 17 wherein these nanoparticles are used against COVID-19.

19. The polyether polymer according to claim 8, wherein end-group fidelity of the polymer in regard to group X and/or in regard to group Y is at least 95%.
